(19) United States
(12) Patent Application Publication
(10) Pub. No.: US 2026/0130946 A1
(43) Pub. Date: May 14, 2026

(54) Ultrapurified Phospholipoproteomic Composition for High-Purity Biomolecular Research and Precision Therapeutics

(71) Applicant: AETHERION GLOBAL LLC, LEWES, DE (US)

(72) Inventor: Ramon E. Gutierrez-Sandoval, Concepcion (CL)

(73) Assignee: AETHERION GLOBAL LLC, LEWES, DE (US)

(21) Appl. No.: 19/264,800

(22) Filed: Jul. 9, 2025

Related U.S. Application Data
(63) Continuation-in-part of application No. 19/088,927, filed on Mar. 24, 2025, now abandoned.
(60) Provisional application No. 63/717,938, filed on Nov. 8, 2024.

(51) Int. Cl. A61K 35/28; C12N 5/078

(52) U.S. Cl. CPC A61K 35/28; C12N 5/0634

(57) ABSTRACT

The present application discloses PLPC-DB, an ultrapurified phospholipoproteomic composition derived from the supernatant of peripheral blood mononuclear cells (PBMCs). It comprises essential phospholipids, bioactive proteins, regulatory peptides, and intercellular signaling factors. The composition exceeds 99% purity through a multi-stage purification process combining high-speed centrifugation and selective ultrafiltration (1-50 kDa), followed by lyophilization. This process ensures structural stability for ≥24 months and inter-batch variability <2%. PLPC-DB supports reproducibility and molecular integrity in research and diagnostic settings. Key components include phosphatidylcholine and phosphatidylserine for membrane stabilization and intracellular signaling; structural and regulatory lipids for membrane biogenesis and immune-relevant components involved in antigen presentation, cytokine modulation, and membrane-associated signaling. These may include structurally defined molecules compatible with immunological evaluation in experimental and non-therapeutic settings. The composition is formulated for bioaccessible delivery via sublingual, endonasal, transdermal, and injectable routes, and is intended exclusively for non-therapeutic use in experimental and translational models.

### CROSS REFERENCE TO RELATED APPLICATIONS

This application is a Continuation in part of Utility Patent application claiming priority to U.S. patent application Ser. No. 19/088,927, filed on Mar. 24, 2025, which in turn claims the benefit of U.S. Provisional patent Application Ser. No. 63/717,938, filed Nov. 8, 2024, both of which are incorporated by reference herein in their entirety.

### COPYRIGHT STATEMENT

A portion of this patent document contains material protected by copyright. The copyright holder grants permission for the facsimile reproduction of this document as it appears in the records of the Patent and Trademark Office. However, all other copyright rights are expressly reserved.

Any trademarks referenced in this disclosure remain the property of their respective owners. The applicant(s) make no claim of ownership over these trademarks.

### BACKGROUND OF THE INVENTION

### 1) Field of Invention

This continuation specifically claims a phospholipoproteomic composition focusing on its immunological profiling, optimized administration routes, and molecular subsets evaluated under controlled biological conditions, excluding therapeutic claims. This innovation pertains to the field of highly purified molecular bioinputs, specifically an ultrapurified phospholipoproteomic concentrate (PLPC-DB). PLPC-DB is derived from supernatants of peripheral blood mononuclear cells (PBMCs) or commercial cell lines and is designed for applications in advanced biomedical research, biotechnological development, and theranostic technologies.

This biotechnological approach extends beyond PBMC-derived supernatants, encompassing cultures from various cell lines, including immature dendritic cells, mature dendritic cells, and antigen-pulsed mature dendritic cells. The ability to source from diverse cellular environments enhances its applicability, particularly in immunology, dendritic cell research, and immune response studies.

By offering a versatile bioactive composition, PLPC-DB supports a wide range of research and development protocols requiring standardized yet adaptable molecular bioinputs for high-precision applications.

### 2) Description of Related Art

One of the primary challenges in biomedical research and biotechnological applications is the development of bioinputs that meet high purity and safety standards while maintaining functional biomolecular integrity. Many compositions derived from cell culture supernatants or biological extracts contain extrinsic proteins, cellular debris, and chemical contaminants that interfere with therapeutic reproducibility, diagnostic accuracy, and biocompatibility in high-precision applications.

The PLPC-DB platform directly addresses these limitations by implementing an ultrapurification process that removes non-specific biomolecules, immunogenic contaminants, and unwanted molecular interactions, ensuring a purity level exceeding 99%. This technological advancement makes it a critical input for next-generation biomedical research, biotechnological innovation, and theranostic applications requiring exceptional standardization and reproducibility.

The current landscape of bioactive phospholipids and proteins is dominated by sources of animal or plant origin, which present significant limitations in research and production environments. These biological materials exhibit batch-to-batch variability, contain residual enzymatic activity and process-related impurities, and often require chemical extraction techniques that degrade functional biomolecules. Furthermore, their application in human and animal models poses risks of cross-reactivity, immunogenic responses, and regulatory constraints, making them unsuitable for high-precision biomedical applications.

PLPC-DB maintains a purity exceeding 99% through a highly controlled purification process, effectively eliminating endotoxins, biological residues, and cellular contaminants. This protocol has been validated in observational studies with 512 patients, confirming its safety and efficacy in real-world clinical environments. Its formulation eliminates the biological variability associated with animal- or plant-derived products, ensuring biocompatibility in advanced immunotherapy and cellular regeneration applications.

While cell culture systems have gained relevance in biomolecule production, current methods for isolation and purification remain inadequate for high-precision applications. Many existing refinement techniques fail to remove intrinsic cellular components, extrinsic proteins, or enzymatic residuals, leading to heterogeneous biomolecular profiles that compromise experimental reproducibility, safety, and regulatory compliance. Moreover, traditional ultrafiltration and centrifugation-based purification often result in protein denaturation, phospholipid degradation, and loss of key bioactive interactions, making them unsuitable for applications requiring intact phospholipid-protein-peptide complexes. PLPC-DB surpasses conventional purification methodologies by integrating ultrafiltration potentially assisted and optimized by artificial intelligence (AI) for selective biomolecule retention. This optimization reduces batch-to-batch variability to less than 2%, a significant advancement over conventional therapies such as CAR-T or monoclonal antibody-based immunotherapies, which exhibit stability variations of up to 15%. Additionally, PLPC-DB offers a modular structure that allows customization of purification parameters depending on the type of bioactive required for specific therapeutic applications.”

Batch-to-batch reproducibility remains a major limitation in biomolecular production. Even minor deviations in phospholipid ratios, bioactive protein concentrations, or structural conformation can significantly impact experimental results, immunomodulatory responses, and therapeutic efficacy. Current phospholipoproteomic purification methods lack the precision and adaptability required to meet the demands of modern biotechnology and pharmaceutical applications. Variations in biochemical stability, lipid-protein-peptide integrity, and process consistency hinder their implementation in preclinical and clinical trials, high-throughput screening, and diagnostic formulations. The PLPC-DB platform resolves this issue by integrating an AI-driven purification system that ensures batch-to-batch reproducibility below 2% variance, establishing a scalable, precision-optimized biomolecular input for biomedical research, immunotherapy, and diagnostic development.

The demand for ultrapure phospholipid-protein complexes in biotechnology, regenerative medicine, and molecular diagnostics continues to increase. These bioinputs must adhere to the following criteria:
High biocompatibility for human and animal research applications.
Molecular uniformity to support reproducibility in multicenter clinical trials and precision therapies.
Functional integrity preservation throughout the purification, storage, and application phases.
Complete elimination of immunogenic and cytotoxic contaminants to ensure regulatory compliance.
   The PLPC-DB innovation not only meets but exceeds these requirements, providing a scalable, standardized, and bioactive phospholipoproteomic composition optimized for next-generation biomedical research and translational applications.

As precision medicine, immunotherapy, and molecular regeneration continue evolving, the necessity for standardized, ultrapure biological inputs becomes evident. The ability to modulate immune responses, drive cellular regeneration, and enhance molecular diagnostics requires bioinputs that serve as both high-fidelity research tools and clinically viable biomolecular therapies. The PLPC-DB system fulfills this dual function by offering a highly adaptable phospholipoproteomic concentrate capable of seamlessly transitioning from preclinical research to regulated therapeutic applications, ensuring uninterrupted continuity from laboratory validation to clinical implementation.

The development of next-generation phospholipoproteomic purification technologies presents transformative opportunities in multiple fields, including:
Cell Biology & Immunology—Advancing immune modulation, antigen presentation, and cytokine profiling.
Regenerative Medicine—Enhancing stem cell differentiation, extracellular matrix remodeling, and tissue engineering.
Molecular Therapeutics—Facilitating targeted phospholipid-based formulations for immunotherapy and precision oncology.
Diagnostic Bioinputs—Providing high-sensitivity, high-specificity biomolecular standards for next-generation assays.
   The PLPC-DB system directly addresses the limitations of traditional bioinput sources and purification methodologies, setting a new benchmark for biomolecular standardization, reproducibility, and therapeutic integration.

The technical specifications, structural innovations, and application spectrum of this invention will be further detailed in the accompanying drawings and descriptions of preferred embodiments, demonstrating the scientific and technological superiority of the PLPC-DB purification system in comparison to conventional methodologies.

The biotechnology and biomedical research sectors face an increasing demand for ultrapurified and standardized bioactive phospholipids and proteins that can be reliably integrated into diverse applications, including regenerative medicine, immunomodulation, and molecular diagnostics. Traditional sources of these biomolecules often introduce contaminants, batch-to-batch variability, and unwanted molecular interactions, limiting their reproducibility and safety in high-precision applications. PLPC-DB directly addresses these limitations by offering a high-purity, contaminant-free, functionally stable phospholipoproteomic composition, optimized for therapeutic and research use in controlled biomedical environments.

The purification and optimization process of PLPC-DB is supported by a computational model that continuously monitors and adjusts key process parameters in real time. This model integrates large datasets from cell culture conditions, supernatant biomolecular content, and ultrafiltration performance, allowing dynamic adjustments in:
Centrifugation speed and duration based on sedimentation efficiency and molecular weight distribution.
Ultrafiltration porosity and pressure settings to optimize phospholipid and protein retention.
pH and temperature regulation to ensure structural integrity and bioactivity of biomolecules.

The AI-driven optimization system operates within a predefined framework, refining purification parameters based on quality control data and pre-established biological thresholds. This approach enhances reproducibility, reduces batch variability to below 2%, and ensures consistent purity exceeding 99%. The algorithm functions as an adaptive monitoring system rather than an autonomous decision-making entity, allowing pre.cise control over production while maintaining compliance with regulatory quality standards

The biotechnology and biomedical research sectors face an increasing demand for ultrapurified and standardized bioactive phospholipids and proteins that can be reliably integrated into diverse applications, including regenerative medicine, immunomodulation, and molecular diagnostics. Traditional sources of these biomolecules often introduce contaminants, batch-to-batch variability, and unwanted molecular interactions, limiting their reproducibility and safety in high-precision applications. PLPC-DB directly addresses these limitations by offering a high-purity, contaminant-free, functionally stable phospholipoproteomic composition, optimized for therapeutic and research use in controlled biomedical environments.

The development of advanced purification methodologies for phospholipoproteomics and bioactive biomolecule extraction represents a critical technological breakthrough, with the potential to eliminate batch inconsistencies, optimize biomolecular functionality, and enhance the applicability of these inputs in precision medicine. Innovations in AI-monitored purification, adaptive ultrafiltration, and multi-stage bioactive molecule refinement can directly impact research and clinical advancements in cell biology, immunology, regenerative medicine, and targeted biopharmaceuticals.

The technological innovations, advantages, and broad applications of PLPC-DB will be further detailed in the accompanying drawings and preferred embodiment descriptions, providing comprehensive insight into the novel purification strategy, biofunctional optimization, and high-reproducibility characteristics that distinguish this invention from conventional biomolecular purification methods.

### SUMMARY OF THE INVENTION

Addressing Limitations in Existing Phospholipoproteomic Purification Methods: The present invention introduces PLPC-DB, an ultrapurified phospholipid-protein-peptide composition designed to meet the highest standards of purity, functional integrity, and biocompatibility. This platform directly addresses the challenges posed by batch-to-batch variability, contamination risks, and structural instability in existing phospholipoproteomic purification technologies. PLPC-DB is formulated as a high-purity bioinput optimized for biomedical research, diagnostic applications, immunotherapy, and precision medicine.

A Breakthrough in Ultrapurified Phospholipoproteomic Composition: PLPC-DB is produced through a rigorously controlled, AI-integrated purification process, ensuring consistency, reproducibility, and regulatory compliance. It is:
Free of animal or plant-derived contaminants, eliminating risks of cross-reactivity and immunogenic responses.
Standardized under strict biotechnological protocols, ensuring compliance with GRAS (Generally Recognized as Safe), ISO 13485 (medical-grade biomanufacturing), and PPH (Patent Prosecution Highway) guidelines.
Tailored for high-sensitivity applications in cellular biology, regenerative medicine, and biomolecular diagnostics.

PLPC-DB Purification Process and Technological Differentiation: The PLPC-DB purification process employs a multi-stage separation system combining advanced centrifugation and ultrafiltration, allowing for the selective retention of bioactive molecules while eliminating contaminants and structural impurities.

The purification process regulates multiple operational parameters, including centrifugation speed (RPM), ultrafiltration pore size (kDa), and sequential cycle optimization, ensuring batch-to-batch reproducibility below 2% variance. These parameters are continuously optimized through artificial intelligence (AI) algorithms, enabling real-time monitoring and process adaptation.

The ultrafiltration system employs membranes with adjustable pore sizes optimized for bioactive protein retention and selective exclusion of lower molecular weight components, preserving a high degree of purity and molecular functionality.

### Centrifugation Parameters:

Speeds exceeding 10,000 RPM, with adjustable cycle regulation.
Dynamic real-time adjustments for optimal separation of phospholipids, proteins, and peptides.
Scalability for high-throughput research and industrial applications.

### Ultrafiltration Membrane Porosity:

10-50 kDa-Retains bioactive proteins, allowing peptides and phospholipids to pass.
3-10 kDa-Selective purification of signaling peptides while allowing lipid passage.
1-5 kDa-Isolation of ultrapure phospholipids with minimal protein interference.

### Process Automation & AI Integration:

AI-driven optimization of purification cycles, ensuring consistency across production batches.
Real-time adaptive process control, monitoring molecular composition fluctuations.
Automated quality control system, ensuring compliance with ISO 13485 and FDA purity standards.
   The methodology ensures purity levels exceeding 99%, eliminating cellular debris, contaminants, and process-induced molecular degradatio

Key Composition Features and Purity Standards: The present invention provides a purified phospholipoproteomic composition containing specific proportions of phosphatidylserine, phosphatidylcholine, and other bioactive molecular components. The composition is characterized by a purity level exceeding 99%, achieved through a multi-stage purification process integrating high-speed centrifugation and adaptive ultrafiltration. This process ensures the removal of non-specific biomolecules, residual cellular debris, and extrinsic contaminants, while preserving the functional integrity and biochemical stability of phospholipids, proteins, and signaling peptides.

PLPC-DB is specifically formulated to be highly biocompatible and structurally stable, making it suitable for a wide range of biomedical applications, including immune response studies, cell signaling modulation, regenerative medicine, and advanced diagnostic methodologies. The high degree of purity and consistency in molecular composition allows for reproducibility across production batches, an essential requirement for multicenter research, clinical development, and biotechnological scalability.

The purification process is precisely controlled to ensure the exclusion of enzymatic activity, unwanted protein aggregates, and process-induced molecular degradation, allowing the final composition to maintain its bioactive properties under various storage and handling conditions. The resulting composition is an optimal platform for translational medicine, molecular diagnostics, and cell-based therapeutic research, supporting its use in next-generation biomedical applications.

PLPC-DB has been designed for registration in multiple jurisdictions due to its compliance with GRAS (FDA), Novel Food (European Union), and NHP (Canada). Its regulatory acceptance is supported by international licensing models, allowing its production under GMP regulations without compromising its scientific and safety foundation. Additionally, the Health Products (Therapeutic Products) Regulations 2016 in Singapore permit non-NCE biotherapeutics to undergo accelerated evaluation, exempting them from conventional preclinical animal testing and phase 1-3 clinical trials if supported by sufficient observational clinical evidence, such as the data validating PLPC-DB.

Expanded Applications and Functional Properties: The purified phospholipoproteomic composition described in this invention demonstrates a broad range of biomedical, therapeutic, and diagnostic applications, extending its utility beyond traditional bioinputs. Due to its high molecular integrity, stability, and functional reproducibility, PLPC-DB serves as a core component in precision research and translational medicine.

Among its primary applications, PLPC-DB is utilized as:
A critical bioinput in biomedical research-Its highly standardized nature makes it an essential component for preclinical studies, immune response assays, and cell signaling investigations. The high degree of batch-to-batch consistency ensures its reliability in controlled experimental settings, multicenter collaborations, and translational research initiatives.
A fundamental biomolecular input for regenerative medicine—The composition's unique profile of bioactive phospholipids and signaling proteins supports cell adhesion, extracellular matrix remodeling, and tissue regeneration. Its purity and functional stability allow for seamless integration into scaffold-based tissue engineering platforms, stem cell differentiation protocols, and cellular microenvironment reprogramming studies.
A next-generation diagnostic reference material-PLPC-DB exhibits exceptional stability and biochemical fidelity, making it an ideal standard for high-precision biomarker assays, mass spectrometry-based proteomic analysis, and immunological profiling. The composition's controlled molecular content enables high sensitivity and specificity in advanced diagnostic technologies, including ELISA, immunofluorescence, and molecular imaging modalities.
A modulator of immune response and cell signaling pathways—The composition has been designed to maintain its bioactivity across diverse biological environments, allowing it to function as an immune modulator in experimental and therapeutic applications. When incorporated into biological systems, cellular assays, or in vivo models, PLPC-DB maintains its molecular integrity, interacts with target cell receptors, and contributes to the regulation of immune activation, inflammation, and regenerative responses.
An advanced formulation platform for biopharmaceutical applications-PLPC-DB's ability to retain functional bioactivity under a range of experimental conditions supports its incorporation into controlled-release therapeutic formulations, bioactive nanocarriers, and synthetic extracellular matrices. The composition's unique physicochemical properties make it highly adaptable to next-generation therapeutic delivery systems, precision-targeted interventions, and customized bioengineered therapies.
Clinical studies have demonstrated that PLPC-DB achieves a tumor response rate of up to 90%, compared to 85% for CAR-T and 70% for monoclonal antibodies. A documented reduction of 78% in IL-10 and 72% in TGF-β has been observed, promoting prolonged immune activation without significant adverse events. Stability studies have shown that PLPC-DB retains 96% of its bioactivity after 12 months of storage, surpassing other immunomodulatory therapies in functional stability.
   Additionally, PLPC-DB can be formulated into various dosage formats, including liquid suspensions, lyophilized powders, and injectable preparations, allowing for optimized handling, stability, and delivery across different biomedical applications. Comparative studies indicate that PLPC-DB exhibits enhanced bioavailability, superior molecular integrity, and greater functional retention compared to conventional bioinputs, making it a leading candidate for integration into future biotechnological, pharmaceutical, and therapeutic advancements.

Patent Scope and Industrial Applications: The present patent encompasses both the composition itself and the purification methodology that ensures its structural integrity, functional activity, and reproducibility across large-scale production. The scope of protection includes:
1. The phospholipoproteomic composition and its molecular specification
   The composition consists of ultrapurified phospholipids, bioactive proteins, and peptides derived from peripheral blood mononuclear cell (PBMC) supernatants or commercial cell cultures.
   The bioactive content is maintained at purity levels exceeding 99%, verified through high-performance liquid chromatography (HPLC), mass spectrometry (MS), and advanced proteomic analyses.
   The composition retains its biofunctional properties under controlled storage, handling, and experimental conditions, ensuring its suitability for research, therapeutic, and diagnostic applications.
2. The AI-driven purification process
   The purification method employs high-speed centrifugation, adaptive ultrafiltration, and dynamic parameter optimization.
   The system continuously monitors and adjusts purification parameters using real-time AI-driven analytics, ensuring batch-to-batch reproducibility below 2% variance.
   The combination of centrifugation, ultrafiltration, and automated quality control mechanisms ensures selective biomolecular retention, contaminant exclusion, and preservation of phospholipid-protein-peptide interactions.
3. The composition's applications in research, diagnostics, and biotechnology
   Biomedical Research-Serves as a high-purity, standardized input for experimental and translational studies.
   Molecular and Cellular Diagnostics-Functions as a high-sensitivity biomolecular standard for disease biomarker detection, cell-based assays, and proteomic research.
   Regenerative Medicine and Cell Therapy-Supports stem cell differentiation, immune modulation, and extracellular matrix interactions in tissue engineering and biofabrication platforms.
   Precision Therapeutics and Drug Delivery-Integrates into biocompatible therapeutic formulations, nanoparticle-based carriers, and bioactive scaffold systems for controlled drug release and molecular targeting.
      The technical innovations, biomedical applications, and commercial viability of this invention establish PLPC-DB as a leading ultrapure phospholipoproteomic composition, setting a new standard in biomolecular precision, research reproducibility, and translational medicine.

The scope of the present invention extends to all embodiments, formulations, and technological advancements derived from the composition and purification methodology described herein, reinforcing its industrial relevance, clinical potential, and long-term impact in biotechnology and pharmaceutical innovation.

Ultrapurified Phospholipoproteomic Composition (PLPC-DB) as a Standardized Biotechnological Input: The present invention is framed within the field of biomolecular bioinputs and focuses on the development of PLPC-DB, an ultrapurified phospholipoproteomic composition designed as a high-precision tool for biomedical research, biotechnological applications, and molecular diagnostics. This standardized bioinput is formulated to meet the highest standards of purity, biocompatibility, and functional integrity, making it an essential component in experimental and translational research.

PLPC-DB is produced through a highly controlled purification protocol, ensuring reproducibility across multicenter research and industrial biomanufacturing. The composition is derived from phospholipids, bioactive proteins, and peptides, obtained through an advanced centrifugation and ultrafiltration process that adheres to GRAS (Generally Recognized as Safe) and ISO 13485 standards. As it is entirely free from animal- or plant-derived sources, PLPC-DB eliminates the risk of immunogenic contamination, making it suitable for a broad spectrum of biomedical applications, including precision medicine, cell therapy, and advanced immunomodulation studies.

The purification system of PLPC-DB integrates adjustable centrifugation and ultrafiltration parameters, allowing for precise molecular fractionation and selective retention of essential bioactives. By combining centrifugation speeds exceeding 10,000 RPM with ultrafiltration membranes featuring adaptable porosity starting at 3 kDa, the process ensures:
Retention of bioactive phospholipids and proteins, while removing unwanted contaminants and low-molecular-weight byproducts.
Preservation of biomolecular integrity, ensuring the stability and efficacy of phospholipid-protein-peptide interactions.
Scalability across research and biopharmaceutical development, allowing for consistent batch-to-batch reproducibility.
   This adaptability makes PLPC-DB a unique and high-value biomolecular tool, specifically designed for cutting-edge applications in molecular medicine, immunotherapy, and regenerative biology.

PLPC-DB Purification Process includes High-Speed Centrifugation and Ultrafiltration Optimization. The purification of PLPC-DB is performed using a multi-step refinement system that combines high-speed centrifugation and ultrafiltration, ensuring the selective separation of phospholipid, protein, and peptide fractions from peripheral blood mononuclear cell (PBMC) supernatants or commercial cell cultures. The purification workflow is designed to regulate multiple processing parameters, including:
Centrifugation speed (RPM) and cycle sequencing.
Ultrafiltration membrane porosity (kDa) to control molecular weight separation.
Dynamic alternation of purification cycles for enhanced bioactive retention.
   To ensure real-time optimization, the purification system integrates artificial intelligence (AI)-driven process control, allowing:
Continuous monitoring and adjustment of purification parameters, based on real-time analysis of the biomolecular composition.
Adaptive process optimization, ensuring the highest levels of purity and bioactive functionality in the final composition.
Automated contamination detection and quality control validation, reducing process variability and increasing batch-to-batch reproducibility below 2% variance.

The ultrafiltration system employs adjustable membrane pore sizes, optimized for:
Retention of high-molecular-weight proteins while excluding lower molecular weight components.
Selective filtration of phospholipids and peptides, ensuring functional bioactivity. Maximization of purity and removal of non-target biomolecules.

### Technological Features of the PLPC-DB Purification System

1. Centrifugation Parameters
   Centrifugation speeds exceed 10,000 RPM, with adjustable cycle regulation. The number of cycles and duration are adaptable based on sample characteristics and purity requirements.
   Dynamic tuning of separation conditions, optimizing the retention of functionally active biomolecules.
2. Ultrafiltration Porosity Optimization
   The ultrafiltration membranes have an adjustable cut-off range of 3 kDa to 30 kDa, providing:
      10-50 kDa-Retention of large bioactive proteins while allowing peptides and phospholipids to pass.
      3-10 kDa-Purification of bioactive peptides, optimizing molecular interaction properties.
      1-5 kDa-Isolation of ultrapure phospholipids, ensuring high structural integrity.
3. Parameter Selection Flexibility
The purification system allows for precise control over RPM, ultrafiltration porosity, and processing duration, ensuring: Customizable separation for specific bioactive profiles.
   Consistency and reproducibility in large-scale applications.
   Minimization of batch-to-batch variation, ensuring stable and high-purity final compositions.
4. Purity and Safety Validation
   The methodology guarantees purity exceeding 99%, validated through: High-performance liquid chromatography (HPLC) and mass spectrometry (MS).
      Regulatory compliance with international safety and biocompatibility standards.
      Comprehensive batch testing for purity, molecular stability, and functional retention.

### Process Advantages and Novelty

The PLPC-DB purification methodology is designed to optimize applicability across multiple research and industrial settings, offering:
Scalability for biopharmaceutical manufacturing and molecular research.
Flexibility for integration into clinical and diagnostic applications.
High reproducibility for preclinical and translational studies.
   The system's unique capacity to maintain functional stability above 99% purity reinforces its value in biomolecular and cellular research, making PLPC-DB a transformative tool for next-generation biotechnology.

Molecular Composition and Functional Stability: In accordance with one aspect of the present invention, PLPC-DB is a purified phospholipoproteomic composition containing a precisely defined percentage of phosphatidylserine, phosphatidylcholine, and other bioactive molecules. The composition achieves purity levels exceeding 99% through a rigorously controlled purification process that employs:
Advanced centrifugation for targeted biomolecule separation.
Ultrafiltration-based fractionation to eliminate contaminants and preserve bioactivity.
Molecular stability optimization to retain function across research, diagnostic, and therapeutic applications.
   This process ensures that PLPC-DB remains a reliable and high-performance bioinput for precision medicine, regenerative research, and high-sensitivity diagnostics.

Expanded Functional Capabilities and Research Applications: The PLPC-DB composition may include additional features, supporting its use in multiple biomedical and biotechnological contexts:
Biomedical Research & Immunology—PLPC-DB is an optimal bioinput for cell signaling studies, immune response assays, and experimental disease models.
Regenerative Medicine & Cell Therapy—Its functional stability makes it suitable for controlled microenvironment modulation and cellular differentiation protocols.
Molecular Diagnostics—Functions as a reference material for proteomic analysis, biomarker screening, and clinical immunoassays.
Bioavailability Enhancement—The composition exhibits higher bioavailability and molecular integrity compared to conventional bioinputs, enabling optimized therapeutic potential.
Targeted Biopharmaceutical Applications—Can be formulated into liquid suspensions, lyophilized powders, and controlled-release systems, supporting next-generation therapeutic interventions.

Scope of Disclosure and Technical Advancements: The foregoing description of PLPC-DB, its purification process, and its applications illustrates key technical and biopharmaceutical advancements, without limiting the scope of the invention. The technical specifications, molecular configurations, and process optimizations described herein are intended to provide a comprehensive framework for the industrial application and continued development of PLPC-DB, reinforcing its long-term impact in biomedical research, regenerative medicine, and precision diagnostics.

Certain operational variables such as centrifugation force (RCF), precise RPM values, incubation times, temperature cycles, pH conditions, buffer compositions, and purification sequences may be fine-tuned depending on the functional target or source cell type. These adjustments are considered proprietary industrial process knowledge and are not disclosed in this application. Their exclusion does not affect the reproducibility, enablement, or clarity of the invention as presented.

In accordance with one aspect of the present disclosure, a purified phospholipoproteomic composition is provided containing a specific percentage of phosphatidylserine, phosphatidylcholine and other bioactives, guaranteeing a purity greater than 99%, obtained through an advanced centrifugation and ultrafiltration process.

Expanded Functional Features and Applications of PLPC-DB: The present invention also encompasses additional functional characteristics and biomedical applications of PLPC-DB, which enhance its utility, bioavailability, and research adaptability. The composition may exhibit one or more of the following features:

### 1. Purification and Processing Integrity

PLPC-DB is obtained through a high-speed centrifugation and ultrafiltration methodology, ensuring:
Complete removal of contaminants, including unwanted cellular debris, residual enzymes, and extraneous proteins.
Preservation of bioactive molecular functionality, preventing degradation during processing.
Controlled separation of phospholipids, proteins, and peptides, optimizing purity while maintaining biomolecular activity.

### 2. Versatility in Biomedical Research and Molecular Applications

PLPC-DB is specifically designed for use in a variety of biomedical, biotechnological, and diagnostic applications, including:
Biomedical research—Serves as a high-purity bioinput for molecular biology, proteomics, and immune modulation studies.
Cell signaling investigations—Acts as a molecular modulator, supporting investigations into intracellular communication pathways.
Immune support protocols—Contributes to adaptive immune regulation, cytokine modulation, and antigen-presenting cell function.
Cellular and molecular development—Functions as a critical biomolecular input for experimental and applied cellular biology.
Biomolecular diagnostics—Serves as a reference standard in proteomic assays, mass spectrometry, and biomarker screening techniques.

### 3. Functional Stability and Biocompatibility in Biological Systems

When incorporated into cell cultures or biological systems, PLPC-DB maintains:
Structural integrity, preventing molecular breakdown under standard storage and usage conditions.
Functional bioactivity, preserving phospholipid-protein interactions and bioactive molecular signaling.
Reproducibility across experimental models, supporting high-throughput research and clinical validation.

### 4. Specific Biochemical and Therapeutic Properties

PLPC-DB demonstrates a range of unique biochemical functions, including:
Modulation of biochemical factors-Facilitates controlled regulation of cellular microenvironments.
Enhancement of in vitro cell viability-Promotes improved cell survival and metabolic activity in preclinical models.
Vehicle for bioactive agent delivery-Functions as a carrier matrix for targeted molecular administration.

### 5. Bioavailability Optimization and Pharmacokinetic Stability

PLPC-DB may include stabilizing excipients or molecular additives to:
Enhance bioavailability, ensuring optimal molecular absorption in research and therapeutic models.
Improve efficacy across different applications, making it suitable for extended use in biomedical formulations.
Extend functional stability, preserving biomolecular activity under diverse storage and handling conditions.

### 6. Cell Signaling and Disease Research Applications

PLPC-DB is a powerful investigative tool for metabolic disorders, neurodegenerative conditions, and immune dysregulation studies. It may:
Modulate specific intracellular signaling pathways, supporting research on:
   Inflammation and immune response.
   Tissue regeneration and wound healing.
   Tumor microenvironment modulation.
Enhance diagnostic biomarker detection, serving as a high-precision reagent in proteomic analysis.
   7. Integration into Diagnostic and Research Systems
   PLPC-DB can be incorporated into:
Diagnostic assays—As a highly stable reference material for disease biomarker screening.
Advanced proteomic studies—Ensuring consistent analytical reproducibility.
Multi-component research formulations—Enhancing the synergistic effects of combined bioactive compounds.

### 8. Adaptability in Formulation and Delivery

PLPC-DB can be formulated in various dosage and delivery formats, including:
Aqueous and buffer-based solutions for cell culture and in vitro applications.
Lyophilized powders, ensuring long-term stability and ease of storage.
Liquid injectable formulations, allowing for direct integration into controlled experimental models.

### 9. Comparative Performance and Competitive Superiority

PLPC-DB exhibits superior efficacy, bioavailability, and safety compared to conventional bioinputs, as demonstrated in:
Preclinical and clinical research models, validating enhanced functional stability and reproducibility.
Comparative efficacy trials, confirming improved biomolecular interactions and cellular compatibility.

Scope of Disclosure and Non-Limiting Nature of Embodiments: The foregoing general description, along with the subsequent detailed description, presents exemplary embodiments that illustrate the principles and applications of the present invention. These descriptions serve as representative examples of the invention's technical scope, functional capabilities, and applicability in biomolecular research and biotechnology. However, they should not be interpreted as limiting in nature, as modifications, adaptations, and alternative implementations may be developed within the spirit and scope of the disclosed technology.

Additional Objectives and Advantages: Additional features, enhancements, and benefits of the present invention will become evident through further examination of the specification, experimental validation, and practical implementation. While some aspects of the invention may be immediately apparent to experts in the field, others will be further elucidated through the detailed descriptions, performance data, and supporting documentation provided herein.

This disclosure anticipates continuous innovation, optimization, and expansion of applications, ensuring that PLPC-DB remains adaptable to evolving research, industrial, and clinical requirements in biotechnology, molecular diagnostics, and regenerative medicine.

Expanded Scope and Advantages of the Invention: Additional objectives and benefits of the present invention will become evident through further examination of the specification, experimental validation, and practical applications. While certain aspects of the invention may be immediately recognizable to experts in the field, others will be further clarified through detailed descriptions, empirical data, and performance assessments provided herein.

The present invention not only advances biomolecular purification methodologies but also expands the functional landscape of ultrapure phospholipoproteomic compositions, supporting their broad applicability in biotechnological, therapeutic, and diagnostic research.

Composition and Applications of PLPC-DB: The PLPC-DB composition is a purified phospholipoproteomic formulation comprising:
a. Phospholipids and bioactive proteins derived from peripheral blood mononuclear cell (PBMC) supernatants, with a purity level exceeding 99%, obtained through an advanced purification system that integrates high-speed centrifugation and ultrafiltration. PLPC-DB is specifically designed to address key challenges in biomedical research, molecular diagnostics, and immune-modulating technologies, offering unparalleled reproducibility, standardization, and biocompatibility.

### 1. Advanced Biomedical and Biotechnological Research

PLPC-DB serves as an essential bioinput for molecular biology, proteomics, and cell signaling studies, facilitating:
Multicenter research in tissue regeneration, biomarker discovery, and precision immunology.
Standardized methodologies for immune support, inflammation research, and therapeutic development.
Comprehensive reproducibility across laboratories and biotechnology institutions, ensuring consistency in translational research outcomes.
   PLPC-DB is fully compliant with regulatory safety standards, allowing safe integration into multicenter investigations and ensuring rigorous reproducibility across different research and biotechnological centers.

### 2. Molecular Diagnostic and Theranostic Technologies

PLPC-DB is optimized for use in high-precision diagnostic assays, enabling:
Enhanced detection of disease biomarkers using ELISA, immunofluorescence, and mass spectrometry.
Improved evaluation of inflammatory and metabolic pathways through quantitative biomolecular profiling.
Standardized reference material for next-generation molecular diagnostics, ensuring high sensitivity and specificity in analytical applications.
   By maintaining exceptional molecular stability, PLPC-DB serves as a high-performance input for diagnostic platforms, ensuring accurate and reproducible biomarker quantification.

### 3. Development of Biomarkers and Immune-Modulating Protocols

PLPC-DB provides a platform for the identification, validation, and implementation of clinical biomarkers, allowing:
Characterization of inflammation and metabolic dysfunction indicators through advanced molecular detection systems.
Design of immune-modulation and cellular signaling protocols, supporting personalized therapeutic strategies.
Optimized molecular response tracking in research and preclinical studies, enabling precision-driven immune therapies.
   Its standardized formulation ensures compatibility with translational research frameworks, allowing direct integration into immunological and metabolic research protocols.

### 4. Theranostic Applications and Metabolic Support

PLPC-DB plays a critical role in theranostic research, bridging the gap between early disease detection and therapeutic interventions, by:
Facilitating cell-signaling-based regenerative medicine strategies.
Supporting advanced theranostic methodologies, integrating diagnostic biomolecular signatures with targeted therapeutic approaches.
Enhancing precision metabolic profiling for targeted treatment formulations, providing a molecularly stable platform for biochemical interventions.
   By ensuring high reproducibility, minimal variability, and absolute purity, PLPC-DB allows for seamless integration into next-generation metabolic, regenerative, and theranostic applications.

Overcoming Traditional Challenges in Biomolecular Purification: Biomedical research has consistently faced challenges in obtaining ultrapure bioinputs free from contaminants, particularly in protein-based formulations. Conventional biomolecular purification strategies-often derived from animal or plant-based materials-introduce:
Biological variability, leading to inconsistent molecular compositions across experimental conditions.
High contamination risks, compromising diagnostic precision and reproducibility. Undesirable batch-to-batch fluctuations, affecting molecular interactions and signaling fidelity.
   PLPC-DB resolves these limitations through a proprietary purification process, ensuring:
Purity exceeding 99%, eliminating immunogenic and xenobiotic contaminants.
Standardized phospholipoproteomic integrity, preserving molecular bioactivity.
Batch-to-batch consistency, ensuring reproducibility across preclinical and translational research models.
   PLPC-DB is specifically developed to comply with FDA GRAS (Generally Recognized as Safe) standards, ensuring:
Regulatory approval for safe use in scientific and diagnostic applications.
Non-animal, non-plant-derived formulation, reducing immunogenic interference.
Compatibility with Novel Food (EU) and NHP (Canada) standards, facilitating global multicenter integration.
   As a validated ultrapure bioinput, PLPC-DB is strategically positioned to be integrated into next-generation biomedical, biopharmaceutical, and diagnostic innovations.
   Its unique molecular formulation and purification integrity make it a superior alternative to conventional bioinputs, allowing for a new standard in high-precision biomedical and diagnostic applications.

Overcoming Challenges in Biomolecular Purification: The PLPC-DB Solution: The biomedical and biotechnological research sectors have long faced significant challenges in obtaining high-purity, contaminant-free protein-based bioinputs with standardized molecular profiles. Traditional purification methods, particularly those derived from animal or plant-based sources, introduce biological variability, inconsistent molecular compositions, and a high risk of contamination, which can significantly interfere with advanced cell signaling studies, molecular diagnostics, and precision biotechnological applications.

PLPC-DB addresses these limitations through an innovative multi-stage purification process that ensures:
Purity levels exceeding 99%, verified through high-performance liquid chromatography (HPLC) and mass spectrometry (MS).
Elimination of all plant- and animal-derived contaminants, reducing immunogenic and biochemical interference.
Batch-to-batch reproducibility below 2% variability, ensuring consistent performance across multicenter research initiatives.

### Advantages of the PLPC-DB Purification Process

Unlike conventional protein extraction and purification techniques, which rely on biological sources that inherently introduce variability, PLPC-DB is derived from controlled supernatants of peripheral blood mononuclear cells (PBMCs) or commercial cell lines, ensuring:
Standardized molecular composition, eliminating variations caused by biological heterogeneity.
High stability across research and diagnostic applications, preventing structural degradation of bioactive phospholipids and proteins.
Regulatory compliance with international safety and purity standards, facilitating its use in global scientific and biotechnological initiatives.

### Regulatory Compliance and Global Research Integration

PLPC-DB is formulated to meet and exceed international safety and biocompatibility regulations, including:
FDA GRAS (Generally Recognized as Safe)—Ensuring high-purity standards for biomedical and diagnostic use.
Novel Food (European Union)—Allowing integration into advanced biomolecular research and therapeutic development.
NHP (Natural Health Products, Canada)—Facilitating multicenter research collaboration across North America and Europe.

By eliminating the limitations associated with conventional bioinputs, PLPC-DB establishes a new benchmark for ultrapure biomolecular compositions, enabling its safe, reproducible, and high-performance application in biotechnology, advanced diagnostics, and translational research.

### BRIEF DESCRIPTION OF THE DRAWINGS

The technical structure, process workflow, and functional advantages of the present invention can be better understood by referring to the accompanying specification and drawings, where reference numerals are used to identify similar components across multiple figures. When a reference numeral is mentioned without an explicit sub-label, it is intended to refer collectively to all related components across different views.

FIG. 1 is a flowchart detailing the sequential steps involved in the purification process of phospholipoproteomic compositions;

FIG. 2 is a diagram showing the PLPC-DB purification workflow including a multi-stage process for ultrapure biomolecular composition;

FIG. 3 is a diagram showing components and applications of the PLPC-DB;

FIG. 4 is a chart of overcoming purification challenges with PLPC-DB;

FIG. 5 is a diagram showing the purification process of the phospholipoproteomic composition;

FIG. 6 is a diagram showing purification and characterization of the PBMC-Derived composition; and

FIG. 7 is a diagram showing the PLPC-DB composition and functional biomolecular components.

Corresponding reference characters within the figures denote functionally equivalent parts in various diagrammatic views. The examples provided in these illustrations are intended to showcase specific embodiments of the invention, clarifying key elements of the purification process, but should not be interpreted as limiting the overall scope of the invention. These representations serve to enhance the understanding of PLPC-DB's innovative methodology, ensuring clarity regarding its structural composition, processing parameters, and biotechnological applications.

### DETAILED DESCRIPTION

Scope and Exemplary Embodiments: While various aspects and features of the invention have been summarized, the following detailed description provides a more in-depth illustration of exemplary embodiments, enabling those skilled in the art to fully understand and apply the disclosed concepts. These examples are provided for explanatory purposes and should not be interpreted as restricting the scope of the invention. Alternative implementations and modifications that remain within the principles and objectives of the invention are considered within its intended scope.

Interpretative Flexibility and Feature Integration: This description presents specific technical details and operational methodologies to enhance comprehension. However, one skilled in the art will recognize that the core principles of the invention can be implemented without necessarily including every detail provided herein.

The disclosed embodiments are not mutually exclusive, meaning:
Features described in one embodiment may be incorporated into other embodiments, creating hybrid or optimized configurations.
No single element or feature should be considered essential to every embodiment, as variations exist that may omit certain components while still remaining within the inventive concept.

Terminological Clarifications and Interpretation: To avoid ambiguity, the following rules apply to the interpretation of terminology in this disclosure: The singular form includes the plural, unless explicitly stated otherwise. The use of “and” and “or” should be interpreted as “and/or”, meaning it covers any combination of the listed elements. The term “including” (as well as “includes” and “included”) is non-exclusive, meaning additional, unspecified elements may also be present. Terms such as “element” or “component” encompass both single-unit and multi-unit structures, unless specifically restricted in context.

Logical Constructs and Conditional Exclusions: The terms “or” and “and/or” are to be interpreted inclusively, meaning they allow for any single element, any combination of elements, or all elements together. For example: “A, B, or C” should be read as “any of the following: A; B; C; A and B; A and C; B and C; or A, B, and C”. An exception to this rule applies only when the specific context inherently excludes certain combinations due to functional constraints or technical incompatibility.

Adaptability of the Invention to Multiple Embodiments: This invention encompasses multiple possible embodiments, and the present disclosure should be interpreted as a representative example of its principles, rather than a limitation to specific implementations. The invention is intended to cover all variations, modifications, and equivalent implementations that align with the disclosed principles and functional objectives.

Definition of PLPC-DB: Throughout this specification, the term PLPC-DB refers to a purified phospholipoproteomic composition, obtained through high-speed centrifugation and ultrafiltration to achieve a purity level exceeding 99%, ensuring molecular stability, reproducibility, and functional bioactivity across multiple applications.

Definition of “kDa” in Molecular Weight Measurements: The term “kDa” (kilodalton) represents a unit of molecular mass measurement, commonly used in biotechnology and proteomics to quantify large biomolecules such as proteins, phospholipids, and peptides. Specifically:

$$1 kDa ( kilodalton ) = 1000 daltons ( Da ) .$$

This unit is essential for classifying biomolecules based on molecular weight, facilitating precise ultrafiltration and biomolecular separation techniques.

These definitions ensure scientific clarity and technical accuracy, supporting the structured interpretation of molecular characteristics, purification parameters, and bioanalytical methodologies utilized in the present invention.

### DEFINITIONS AND MOLECULAR CHARACTERIZATION OF PLPC-DB

Definition of “kDa” in Molecular Mass Measurement: The term “kDa” (kilodalton) is a standard unit for measuring the molecular mass of large biomolecules, including proteins, phospholipids, and peptides. In this context:
“k” represents 1000, meaning 1 kDa=1000 daltons (Da).
Dalton (Da) is an atomic mass unit (AMU) used to define the molecular weight of biomolecules.
Kilodaltons are essential in proteomics and molecular biology, as they provide a convenient and scalable measurement framework for classifying biomolecules by mass.
   This unit plays a critical role in biotechnological purification and separation processes, such as ultrafiltration, where molecular size cut-offs (e.g., 3 kDa, 10 kDa, 30 kDa, 50 kDa) are used to retain or exclude specific biomolecular fractions.

Purified Supernatant Phospholipoproteomic Composition (PLPC-DB) and Its Applications: PLPC-DB is a purified phospholipoproteomic composition that may be derived from peripheral blood mononuclear cells (PBMCs) or other biotechnological sources. This composition is enriched with essential bioactive molecules, including:
Phospholipids, which are fundamental for cell membrane structure and intercellular signaling.
Bioactive proteins, which contribute to immune regulation, tissue regeneration, and biochemical stability.

### Purification Process and Functional Advantages

PLPC-DB is obtained through an advanced purification methodology, combining:
High-speed centrifugation, which isolates phospholipids, proteins, and peptides while removing unwanted cellular components.
Precision ultrafiltration, ensuring selective molecular fractionation, eliminating contaminants, and preserving bioactive function.

### Applications in Biomedical and Biotechnological Research

The high level of purity and molecular integrity of PLPC-DB makes it an ideal bioinput for:
Cell regeneration studies, where its phospholipid-protein interactions enhance cellular repair and differentiation.
Immune modulation research, supporting the development of immunotherapeutics and precision medicine applications.
Advanced therapeutic development, facilitating drug delivery systems, controlled-release formulations, and regenerative biomaterials.
   The present disclosure further encompasses methods for producing PLPC-DB-based immune support protocols and customized phospholipoproteomic formulations, extending its utility in molecular and cellular therapies.

Molecular Components of the Purified Phospholipoproteomic Composition: PLPC-DB is composed of a diverse range of bioactive molecules, primarily derived from PBMC supernatants, which serve as a rich source of phospholipids, proteins, and signaling peptides. These molecules play critical roles in multiple biological functions, including:
Cell regeneration—Supporting stem cell differentiation, tissue repair, and extracellular matrix formation.
Immune modulation—Facilitating cytokine signaling, macrophage polarization, and adaptive immune responses.
Intercellular communication—Enhancing cell-to-cell interaction and biochemical network regulation.
   PLPC-DB's ultrapure and standardized composition ensures its compatibility with diverse experimental and therapeutic models, providing a high-value molecular tool for research and translational medicine.

Key Phospholipid Components: Phosphatidylcholine and Phosphatidylserine: The phospholipid fraction of PLPC-DB includes:
Phosphatidylcholine (PC)—A primary component of cell membranes, essential for: Maintaining cellular structural integrity.
   Serving as a precursor for critical second messengers in cell signaling.
   Regulating lipid metabolism and homeostasis.
Phosphatidylserine (PS)—A key molecule in apoptosis and immune signaling, responsible for:
   Facilitating phagocytic clearance of apoptotic cells.
   Modulating immune cell function, including T-cell activation.
   Enhancing cellular membrane fluidity and vesicle formation.
      The inclusion of these phospholipids in PLPC-DB strengthens its applications in regenerative medicine, neurobiology, and immune system modulation, making it a versatile tool in biomedical research.

Bioactive Protein Components: Integrins and Tetraspanins: PLPC-DB also contains critical bioactive proteins, such as:
Integrins, which are involved in:
   Mediating cell adhesion to the extracellular matrix.
   Regulating intracellular signaling cascades related to immune responses and wound healing.
   Supporting tissue remodeling and regenerative processes.
Tetraspanins, which play a role in:
   Facilitating intercellular interactions and cellular migration.
   Modulating immune cell activation and differentiation.
   Organizing membrane microdomains to regulate signal transduction.
      The presence of these bioactive proteins in PLPC-DB enhances its potential for applications in immunotherapy, cancer research, and regenerative tissue engineering.

Role of Intercellular Signaling Molecules in PLPC-DB: PLPC-DB also contains intercellular signaling molecules, which serve as biochemical mediators in complex cellular networks. These molecules:
Facilitate cell-to-cell communication, ensuring coordinated immune responses and metabolic regulation.
Enhance the study of cellular microenvironments, improving understanding of molecular interactions in disease pathology.
Expand its utility in advanced biotechnology, supporting targeted drug development and personalized medicine approaches.
   By maintaining functional stability and molecular fidelity, PLPC-DB optimizes experimental reproducibility and therapeutic efficacy, reinforcing its position as a critical biomolecular input in next-generation biotechnology research.

### Scientific and Biotechnological Applications of PLPC-DB

### Versatility of PLPC-DB in Scientific and Biotechnological Applications

The PLPC-DB composition is designed to support a broad spectrum of research fields and biotechnological innovations, making it an essential standardized bioinput for advanced scientific applications.
Biomedical Research & Molecular Studies—PLPC-DB serves as a fundamental component in laboratories investigating cell regeneration, immune modulation, and other key biological processes.
Development of Novel Biomolecular Protocols—The composition can be customized for immune support protocols and molecular modulation, particularly in regenerative medicine, advanced pharmacology, and personalized medicine.
Veterinary and Aquaculture Research—The non-animal, non-plant-derived formulation enables studies of cellular and molecular interactions in animal models, without the risks of biological incompatibility or cross-species reactivity.
   The high reproducibility and purity of PLPC-DB ensure compatibility across diverse biological systems, supporting both human and veterinary biomedical investigations.

Compositional Customization for Targeted Research Applications: The molecular composition of PLPC-DB is adjustable to accommodate the specific needs of various research areas. The formulation may include precise percentages of phosphatidylserine, phosphatidylcholine, and other bioactive molecules, allowing for optimization in different experimental settings.
Cell Regeneration and Tissue Engineering Studies-A higher concentration of phospholipids such as phosphatidylserine (PS) and phosphatidylcholine (PC) may enhance:
   Membrane stability and integrity, facilitating cell repair and regeneration.
   Cell proliferation and differentiation, making PLPC-DB a valuable tool for tissue engineering and regenerative medicine.
   Enhanced wound healing and neuroprotective applications, ensuring functional recovery in degenerative and injury-related conditions.
      This customization makes PLPC-DB an essential component for preclinical and translational research in regenerative medicine.

Optimization for Immune Modulation and Cellular Interactions: PLPC-DB can be tailored to prioritize bioactive protein content, making it highly effective for immune response and cellular communication studies.
Immunological and Immune-Modulatory Research—The inclusion of a higher proportion of bioactive proteins, such as integrins and tetraspanins, enhances:
   Cell adhesion mechanisms, regulating immune response activation and inhibition.
   Intercellular communication, ensuring cellular coordination in immune system modulation.
   Signal transduction efficiency, improving studies on antigen presentation and T-cell activation.
      This makes PLPC-DB a valuable tool for immunotherapy development, vaccine research, and autoimmune disease investigations.

Customization for Intercellular Signaling Studies: PLPC-DB can also be formulated to enhance intercellular signaling molecule concentrations, ensuring its applicability in advanced molecular biology and biopharmaceutical research.
Intercellular Communication & Signaling Pathway Studies—Adjusting the composition to increase the presence of bioactive signaling molecules can:
   Facilitate targeted investigations into cellular differentiation and immune response regulation.
   Support inflammatory disease research, optimizing anti-inflammatory and immunosuppressive drug discovery.
   Enhance therapeutic modulation studies, allowing researchers to develop novel biologics and signaling-based therapies.
      This compositional flexibility supports biotechnology-driven precision medicine applications, making PLPC-DB an indispensable resource for pharmaceutical development.

Bioavailability Optimization and Delivery Efficiency: PLPC-DB can be formulated to maximize bioavailability, ensuring that bioactive components are efficiently absorbed and utilized in experimental and therapeutic models.
Enhanced Absorption and Stability—The formulation can be modified to:
   Increase bioactive solubility, improving delivery efficiency in biological systems.
   Extend functional stability, preventing biodegradation in high-sensitivity applications.
   Improve molecular retention and controlled release, allowing optimized pharmacokinetics in therapeutic applications.
      These adaptations enhance PLPC-DB's application in targeted drug delivery, biomaterial coatings, and bioactive scaffolds for tissue engineering.

Purification Process: High-Speed Centrifugation and Ultrafiltration Optimization: PLPC-DB is produced through an advanced purification process that integrates:
1. High-Speed Centrifugation for Molecular Fractionation
   Supernatants derived from PBMC cell cultures are subjected to centrifugation exceeding 10,000 RPM.
   Dynamic cycle control allows for:
      Precise molecular separation based on density and biomolecular properties.
   Scalable processing for large-volume production.
   Retention of bioactive molecules with minimal degradation.
2. Ultrafiltration with Adjustable Membranes
   The isolated bioactive components undergo sequential ultrafiltration, using membranes with a minimum cut-off size of 3 kDa.
   Membrane porosity is adaptively adjusted to retain:
      High-molecular-weight proteins while excluding smaller contaminants.
      Specific phospholipids and peptides, ensuring selective fractionation.
      Bioactive molecules with optimal structural integrity, maintaining functional reproducibility across production batches.
3. Integration of Process Adaptability & Quality Control
   AI-driven purification adjustments allow for:
      Real-time process monitoring and optimization.
      Automated molecular integrity validation, reducing batch-to-batch variability below 2%.
      Standardized bioactive composition, ensuring purity exceeding 99%.
         By combining high-speed centrifugation, ultrafiltration, and adaptive purification control, PLPC-DB ensures superior bioactive integrity, functional stability, and broad biomedical applicability, making it an unrivaled biomolecular tool for next-generation research and therapeutic development.

### High-Purity Ultrafiltration and Formulation of PLPC-DB

Optimization of Ultrafiltration for Biomolecular Purity: The purification process of PLPC-DB ensures the precise separation of bioactive molecules through a sequential ultrafiltration system equipped with adjustable membranes featuring a minimum cut-off size of 3 kDa. This system enables:
Selective retention of high-molecular-weight bioactive components while excluding low-molecular-weight contaminants.
Precise tuning of membrane porosity, ensuring maximum retention of phospholipids, proteins, and signaling peptides.
High-efficiency contaminant removal, eliminating residual cellular debris and process-related impurities.

The combination of high-speed centrifugation and adaptive ultrafiltration ensures that the final composition achieves purity levels exceeding 99%, maintaining functional reproducibility across different production settings. This rigorous purification strategy guarantees:
Absolute molecular integrity, ensuring stable biochemical activity.
Reproducibility across research and production batches, making PLPC-DB suitable for biomolecular research and therapeutic applications.
Scalability for industrial and laboratory use, allowing its integration into cellular and molecular development protocols.

Alternative Cell Line Supernatants for Expanded Applications: While peripheral blood mononuclear cells (PBMCs) are a primary source for PLPC-DB, the purification process can also be adapted to other cell-derived supernatants, expanding the biological applications of the composition.

### Cell-Derived Supernatants for Enhanced Functional Properties

Mesenchymal Stem Cell (MSC) Supernatants—These supernatants are rich in growth factors and cytokines, making them highly beneficial for regenerative medicine applications, including:
   Tissue engineering—Enhancing cell proliferation and extracellular matrix formation.
   Wound healing and fibrosis prevention—Supporting epithelial and connective tissue repair.
   Immunomodulation—Regulating inflammatory responses and adaptive immunity.
Epithelial Stem Cell (ESC) Supernatants—These supernatants contain bioactive molecules essential for epithelial cell differentiation and tissue barrier function, enabling:
   Research on skin regeneration and dermatological applications.
   Studies in barrier function regulation and epithelial integrity.
   Investigation of cellular signaling pathways in epithelial-derived disorders.
      By integrating these alternative cell line supernatants into the purification workflow, diverse purified phospholipoproteomic compositions can be generated, each tailored for specific therapeutic and research applications.

Formulation and Long-Term Storage of PLPC-DB: To ensure optimal usability, stability, and transportability, PLPC-DB can be formulated in three distinct formats, each designed for different research and industrial applications:
1. Fresh Liquid Vial
   Preserves bioactive properties in their natural state.
   Ideal for immediate use in live-cell assays and direct functional studies.
   Ensures high bioactivity retention for rapid applications.
2. Cryopreserved Format
   Maintains long-term stability through low-temperature preservation techniques.
   Suitable for medium- to long-term studies where bioactive components must be maintained without degradation.
   Protects molecular integrity without significant loss of functionality.
3. Lyophilized (Freeze-Dried) Format
   Provides a stable, easily transportable powdered form.
   Ensures extended shelf life and structural integrity, ideal for:
      Long-term storage.
      Transport across diverse laboratory and industrial environments.
      Use in biomaterial formulations, including hydrogel-based carriers and scaffold systems.

### Lyophilization Process and Bioactivity Preservation

The lyophilization (freeze-drying) process involves:
1. Freezing the composition at ultra-low temperatures.
2. Reducing atmospheric pressure, allowing frozen water to sublimate directly from solid to gas phase.
3. Preventing molecular degradation, maintaining the composition's structural and functional properties.
   By eliminating moisture while preserving biomolecular stability, this method ensures:
Minimized degradation risks during long-term storage.
Improved bioactive stability across research conditions.
Extended usability in experimental, therapeutic, and biotechnological applications.

Reconstitution and Functional Application of Lyophilized PLPC-DB: The lyophilized form of PLPC-DB can be easily reconstituted in suitable solvents such as:
Distilled water—For cell-based assays and molecular research applications.
Saline buffer solutions—Ensuring physiological compatibility for in vivo and ex vivo studies.
   The reconstitution process can be performed immediately before use, ensuring that:
The bioactive molecules remain in their most potent state for experimental applications. Functional integrity is preserved, making it ideal for high-precision research in cellular biology, immunology, and regenerative medicine.

### Reconstitution and Functional Application of Lyophilized PLPC-DB

The lyophilized form of PLPC-DB can be easily reconstituted in suitable solvents such as:
Distilled water—For cell-based assays and molecular research applications.
Saline buffer solutions—Ensuring physiological compatibility for in vivo and ex vivo studies.
   The reconstitution process can be performed immediately before use, ensuring that:
The bioactive molecules remain in their most potent state for experimental applications. Functional integrity is preserved, making it ideal for high-precision research in cellular biology, immunology, and regenerative medicine.

Advantages of the Freeze-Dried Formulation: The lyophilized (freeze-dried) version of PLPC-DB offers several advantages over conventional liquid formulations:
1. Extended Stability
   The freeze-dried form enhances long-term preservation, preventing molecular degradation over time.
   No refrigeration dependency, reducing storage complexity and logistics costs.
2. Ease of Transport
   Minimizes contamination risks, as the process inactivates potential microbial contaminants (e.g., bacteria, fungi).
   Ensures biosecurity compliance for international research collaborations.
3. Application in Global Research Settings
   Can be safely transported and stored for future use, without significant loss of bioactivity.
   Supports multicenter studies requiring consistency and controlled distribution.
      By integrating lyophilization as a standard formulation option, PLPC-DB ensures its reliability, scalability, and accessibility for advanced biotechnological applications.

Packaging and Handling of Lyophilized PLPC-DB: To facilitate researcher-friendly usage and precision dosing, the lyophilized composition of PLPC-DB is packaged in:
Pre-measured individual vials—Containing a defined amount of the composition, enabling accurate measurement and experimental consistency.
Sterile, sealed containers—Preventing cross-contamination and ensuring bioactive stability.
   Each package may also include:
Detailed reconstitution instructions, specifying optimal dilution ratios, storage guidelines, and usage protocols.
Guidelines for maintaining molecular integrity, ensuring consistent performance in experiments.
   By optimizing packaging, handling, and usability, PLPC-DB becomes a versatile and standardized bioinput, enhancing efficiency in molecular and cellular research.

### Scientific, Biotechnological, and Diagnostic Applications of PLPC-DB

Positioning of PLPC-DB as a Standardized Molecular Bioinput: PLPC-DB is a high-purity phospholipoproteomic composition, specifically designed for biomedical research, molecular diagnostics, and applied biotechnology. As part of the field of purified molecular bioinputs, this invention provides a safe, reproducible, and internationally compliant biomolecular platform, ensuring standardization across diverse scientific and clinical applications.

PLPC-DB is engineered to support advanced research and translational studies, enabling biotechnology researchers and developers to overcome key challenges in bioactive molecule purification, reproducibility, and regulatory compliance. The scope of this invention includes:

Preclinical and Clinical Research Applications: PLPC-DB has been designed for safe and effective use in preclinical and clinical studies, supporting both in vitro and in vivo experimental models. By minimizing biological variability and ensuring functional consistency, PLPC-DB enhances the reproducibility of experimental outcomes, making it a critical input in precision medicine, drug development, and therapeutic research.
Preclinical Research—Used in cell-based assays, immune modulation studies, and regenerative medicine models.
In Vivo Applications—Ensures reproducibility in animal models, supporting drug screening, tissue engineering, and translational research.
Clinical Validation Studies—Facilitates biomarker discovery and diagnostic standardization in multicenter trials and therapeutic evaluations.

### Standardization for Multicenter Diagnostic Technologies

PLPC-DB is optimized for integration into high-sensitivity diagnostic platforms, ensuring precision, reproducibility, and analytical robustness across multicenter research environments.
Biomarker Detection—Enables reliable identification of inflammatory, oncological, and metabolic disease markers.
High-Throughput Analytical Techniques—Compatible with ELISA, immunofluorescence, and mass spectrometry, ensuring high diagnostic accuracy.
Inter-Laboratory Standardization—Facilitates global harmonization of diagnostic assays, improving comparability of research data across institutions.
   The standardized nature of PLPC-DB ensures consistency in data acquisition and validation, making it a cornerstone for molecular diagnostics and biomarker-driven therapeutic strategies.

### PLPC-DB Application Areas

Advanced Biomedical and Biotechnological Research: PLPC-DB serves as a high-purity bioinput for research in molecular biology, proteomics, and cell signaling, with applications including:
Tissue regeneration studies, providing structural phospholipids essential for cell adhesion and differentiation.
Biomarker development, enabling precision detection of disease-specific molecular signatures.
Immune support and cellular modulation, ensuring functional stability in immunological and regenerative applications.
   By offering a standardized and reproducible biomolecular input, PLPC-DB is widely applicable in multicenter research initiatives and therapeutic innovations.

Molecular Diagnostic and Theranostic Technologies: PLPC-DB optimizes biomarker detection and diagnostic precision, supporting next-generation molecular diagnostic frameworks.
Enhances ELISA-based biomarker screening, improving sensitivity and specificity in disease detection.
Supports immunofluorescence-based diagnostics, enabling high-resolution immune profiling and cellular interaction studies.
Advances mass spectrometry-driven proteomics, ensuring accurate molecular characterization of bioactive components.
   By integrating PLPC-DB into theranostic workflows, researchers can bridge the gap between disease detection and targeted intervention, enhancing personalized medicine strategies.

Development of Biomarkers and Immune-Modulating Protocols: PLPC-DB is an ideal platform for the development of clinical biomarkers, providing a highly reproducible and functionally stable composition for:
Inflammation and immune response profiling, ensuring accurate assessment of cytokine and chemokine activity.
Cancer biomarker validation, supporting early detection and targeted therapy research.
Metabolic disorder diagnostics, optimizing biomarker-based evaluations in endocrine and metabolic disease studies.
   Additionally, PLPC-DB contributes to the development of immune support and molecular modulation protocols, ensuring reliable and scalable therapeutic applications.

Theranostic Applications and Metabolic Support: PLPC-DB plays a key role in theranostic innovations, combining diagnostic and therapeutic functionalities to enable:
Early disease detection, providing a highly stable biomolecular framework for precision diagnostics.
Cell signaling-based therapy development, ensuring biochemical fidelity in translational medicine studies.
Tissue regeneration protocols, supporting targeted therapeutic applications in regenerative medicine.
   PLPC-DB's purified and functionally stable composition ensures its viability in both diagnostic and therapeutic formulations, making it an optimal input for metabolic support studies and drug development pipelines.

### Stabilization and Formulation Enhancements

Lyophilized Formulation for Extended Stability and Performance: In certain embodiments, the lyophilized form of PLPC-DB may be enhanced by the addition of stabilizers and excipients, further improving its structural integrity and bioactivity retention.
Lyoprotectants (e.g., sugars, polymers) may be incorporated to:
   Prevent degradation during freeze-drying.
   Maintain structural and functional stability over prolonged storage periods.
   Enhance reconstitution efficiency for immediate usability in research and therapeutic applications.
      These stabilization strategies ensure that PLPC-DB maintains its molecular fidelity, enabling reliable long-term storage and transportation.

### Summary of the Advantages of the Lyophilized Format

The lyophilized version of PLPC-DB offers a highly versatile and convenient formulation, ensuring:
Extended shelf life and stability, minimizing bioactive degradation under variable conditions.
Ease of transport and storage, reducing dependency on refrigeration or specialized handling.
Rapid and controlled reconstitution, enabling on-demand preparation for immediate experimental or clinical use.
   By ensuring the long-term integrity of the phospholipoproteomic composition, the lyophilized form of PLPC-DB is optimized for widespread global application in scientific research, biotechnology, and molecular diagnostics.

### Compositional Characterization, Functionality, and Regulatory Compliance of PLPC-DB

Molecular Composition and Biological Functionality of PLPC-DB: The PLPC-DB formulation is distinguished by its exceptional purity, bioactive molecular stability, and functional relevance in cellular and regenerative processes. The composition includes:
Essential Phospholipids—Comprising phosphatidylserine (PS) and phosphatidylcholine (PC), which:
   Support cell membrane stability, repair, and regeneration.
   Facilitate membrane fluidity and intracellular signal transduction.
   Act as precursors for secondary messengers in cell signaling pathways.
Bioactive Proteins—Key mediators of cellular communication and biochemical signaling, crucial for:
   Cellular adhesion and differentiation.
   Regulation of immune response pathways.
   Signal transduction processes in developmental and regenerative systems.
Peptides—Functionally specialized biomolecules that:
   Optimize the transport and delivery of biochemical signals.
   Regulate homeostatic mechanisms in immune modulation.
   Enhance intercellular signaling cascades for tissue regeneration and repair.
      The purity level exceeding 99% ensures maximal bioactive retention and safety, making PLPC-DB an essential tool for biomedical research, regenerative medicine, and molecular diagnostics.

The Bioactive Component Analysis and Quantification: The PLPC-DB formulation comprises various biomolecular categories, each contributing to its biological efficacy and scientific applicability. The table below summarizes the biochemical composition, its functional role, and the corresponding analytical techniques used for characterization.

### Component Categories and Functional Characterization

| | | Analytical | Estimated Total |
|---|---|---|---|
| Category | Biological Function | Methodology | Amount in 5 mL |
| Cellular | Facilitate intercellular | ELISA and Mass | 150-2000 ng |
| Communication | signaling, acting as key | Spectrometry | |
| Peptides | biochemical mediators in | | |
| | homeostasis and regeneration. | | |
| Structural and | Essential for cell membrane | Gas | 1000-5000 ng |
| Regulatory | biogenesis, intracellular signal | Chromatography- | |
| Lipids | transduction, and modulation of | Mass | |
| | membrane fluidity. | Spectrometry | |
| | | (GC-MS) | |
| Adhesion and | Mediate cell-to-cell interactions | Flow Cytometry | 110,000- |
| Signaling | and immune response | and Western Blot | 220,000 |
| Proteins | coordination, essential for | | molecules |
| | extracellular matrix dynamics. | | |
| Bioactive | Modulate immune response, | ELISA and | 3000-12,000 pg |
| Cellular | inflammation, and tissue repair, | Cytokine Bead | |
| Regulation | regulating immune homeostasis | Array (CBA) | |
| Factors | and inflammatory resolution. | | |
| Phospholipids | Provide structural stability to | High-Performance | 1000-2500 ng |
| | cellular membranes and act as | Liquid | |
| | secondary messengers in signal | Chromatography | |
| | transduction pathways. | (HPLC) | |
| Biochemical | Include metabolites and | Mass | 700-3000 ng |
| Support Factors | hormones that regulate | Spectrometry and | |
| | metabolic homeostasis and | HPLC | |
| | stress adaptation responses. | | |

This quantitative and functional profiling reinforces PLPC-DB's precision, reproducibility. and standardization, ensuring its application across diverse experimental and therapeutic settings.

### Regulatory Compliance and Formulation Safety

Alignment with International Safety Standards

PLPC-DB is formulated in strict compliance with international regulatory frameworks, ensuring its safe integration into scientific research and biotechnological applications. Key regulatory references include:
21 CFR Part 170, Subpart A (§ 170.3)(U.S. FDA)—Establishing safety parameters for bioactive ingredients naturally present in human biology.
EC Regulation No. 258/97 on Novel Food (European Union)—Defining criteria for safe inclusion of bioactive compounds in research and nutraceutical applications.
NHP (Natural Health Products) Regulations, Part 1, Section 2 (Canada)—Ensuring compliance with recognized safety profiles for biomolecular compositions.
   The components of PLPC-DB align with regulatory safety guidelines, as:
They are naturally occurring in human physiology and do not exhibit toxicological risk at the prescribed concentrations.
The exposure levels remain within physiological limits, ensuring non-pharmacological safety.
The biomolecular categories defined in the formulation correspond to essential biological functions, eliminating the need for detailed breakdowns of individual subcomponents, as per 21 CFR Part 170.
   This regulatory alignment provides a legally and scientifically substantiated foundation, ensuring PLPC-DB's acceptance as a safe and standardized biomolecular input for biomedical research, biotechnology, and diagnostic applications.

### Formulation Justification and Safety Considerations

PLPC-DB's composition and molecular structure are designed to:
Support homeostatic and regenerative processes-Ensuring biocompatibility without inducing pharmacological responses.
Maintain concentrations within physiological norms-Aligning with 21 CFR § 170.3(f) on safe exposure levels.
Facilitate international regulatory approval-Enabling its use across multiple research and therapeutic domains.
   By adhering to established safety parameters and maintaining functional biomolecular integrity, PLPC-DB is a scientifically and regulatory-compliant solution for next-generation biotechnological research.

### Purification Process, Functional Characterization, and Biotechnological Applications of PLPC-DB

Variability and Adaptability in Biomolecular Quantification: The estimated concentrations of bioactive components in PLPC-DB are approximate values, subject to variations based on cell culture conditions, activation states, and extraction methodologies. The inherent biological variability of cellular supernatants can influence:
The relative abundance of bioactive phospholipids, proteins, and peptides. The composition of extracellular signaling molecules, particularly in immunomodulatory studies.
The final bioactive profile, requiring precise optimization for specific research applications.

Optimization for Detection Sensitivity: For compounds present at low concentrations, additional biomolecular concentration techniques may be required to enhance detectability, such as:
Affinity purification and molecular enrichment techniques.
Ultrafiltration and selective molecular fractionation.
Advanced mass spectrometry and high-sensitivity ELISA assays.
   These approaches ensure accurate detection and quantification, allowing PLPC-DB to meet the highest standards for biomolecular research and therapeuticKEY PURIFICATION STEPS IN PLPC-DB MANUFACTURING
   The PLPC-DB purification protocol follows a multi-stage, highly optimized methodology that guarantees:
Selective molecular fractionation.
Removal of unwanted cellular debris and contaminants.
Preservation of bioactive molecules in their functional state.
Batch-to-batch reproducibility exceeding 99% purity.
1. Advanced Centrifugation for Efficient Molecular Separation
   Centrifugation at speeds exceeding 10,000 RPM, ensuring the:
      Elimination of non-target cellular components.
      Separation of phospholipids, proteins, and peptides based on density gradients.
      Preservation of bioactive integrity without shear-induced degradation.
   Adjustable centrifugation parameters:
      Cycle number and duration are optimized based on the sample's biomolecular composition.
      Purity objectives dictate the refinement process, ensuring high selectivity.
2. Selective Ultrafiltration for Molecular Precision
   PLPC-DB undergoes stepwise ultrafiltration, allowing for fine-tuned molecular separation based on size and functional properties.
Membrane Porosity Adjustments:
   10-50 kDa-Retains large proteins, excluding peptides and phospholipids.
   3-10 kDa-Purifies peptides while allowing phospholipid passage.
   1-5 kDa-Isolates phospholipids and eliminates high-molecular-weight proteins.
      This customizable purification system ensures:
Consistent purity levels exceeding 99%.
Functional reproducibility across different production batches.
Optimal molecular retention for biomolecular applications in research and therapeutic fields.

Purification Process and Molecular Fractionation: The PLPC-DB purification system is based on a multi-stage, highly controlled methodology, ensuring the selective fractionation of biomolecules, removal of contaminants, and retention of bioactive integrity. This process is characterized by the following steps:
1. High-Speed Centrifugation for Molecular Separation
   Performed at speeds exceeding 10,000 RPM, allowing for:
      Efficient removal of unwanted cellular components.
      Separation of phospholipids, proteins, and peptides based on density gradients.
      Preservation of bioactive integrity without shear-induced molecular degradation.
   The number of cycles and duration are optimized for each sample type, ensuring precision in biomolecular isolation and retention.
2. Selective Ultrafiltration for Molecular Precision
   PLPC-DB undergoes stepwise ultrafiltration, allowing for fine-tuned molecular separation based on size and functional properties. The ultrafiltration system includes adjustable membranes with porosity ranging from 3 kDa to 50 kDa, enabling:
Retention of large proteins using membranes of 10 to 30 kDa, excluding lower molecular weight peptides and phospholipids.
Selective purification of peptides with cut-off sizes between 3 and 10 kDa, retaining proteins while allowing passage of phospholipids.
Separation of phospholipids using 1 to 5 kDa membranes, ensuring isolation from high-molecular-weight proteins and peptides.

This adaptable purification system ensures:
Purity levels exceeding 99%, eliminating all contaminants and cellular debris.
High functional reproducibility across research and production batches.
Optimization for specific biomolecular applications, including biotechnology, regenerative medicine, and diagnostics.

Ensuring Biocompatibility and Safety in Research Applications: The PLPC-DB purification process results in a bioinput that is entirely free from direct cellular manipulation, thereby:
Enhancing biosafety standards for preclinical and clinical research applications.
Eliminating genetic variability concerns associated with animal- and plant-derived biomolecules.
Ensuring compliance with regulatory requirements, facilitating its use in high-precision biotechnology research and therapeutic applications.
   This high level of biocompatibility and molecular integrity ensures PLPC-DB's suitability for advanced research protocols, translational medicine, and multicenter diagnostic investigations.

Variability in Bioactive Concentration and Analytical Considerations: The concentrations of specific bioactive molecules within PLPC-DB may fluctuate depending on:
Cell type-specific expression profiles and culture conditions.
Cellular activation states influencing phospholipid and protein release.
Optimized extraction and enrichment methodologies for targeted biomolecular applications.
   For biomolecules present at low concentrations, additional detection methodologies may be required, including:
Ultrafiltration-based molecular fractionation.
Affinity chromatography for specific bioactive enrichment.
Advanced high-sensitivity mass spectrometry techniques.

Optimization of Low-Concentration Compound Detection: Certain bioactive molecules present in PLPC-DB may exist at low concentrations, making their detection and quantification challenging under standard analytical conditions. To address this, additional molecular concentration and enrichment techniques may be applied, ensuring precise identification of these compounds.

Key approaches for enhancing detection sensitivity include:
Ultrafiltration-Based Molecular Fractionation
   Selective retention of low-abundance peptides and phospholipids using precision ultrafiltration membranes.
   Optimized separation of phospholipid-protein-peptide complexes based on molecular weight and solubility properties.
Affinity Chromatography for Molecular Enrichment
   Isolation of specific bioactive proteins and signaling molecules based on ligand-binding affinities.
   Enhanced purification of low-abundance immune-modulatory factors to improve analytical sensitivity.
Advanced Mass Spectrometry Techniques
   High-sensitivity mass spectrometry (MS/MS and MALDI-TOF) for the precise quantification of peptides, phospholipids, and bioactive regulatory molecules. Stable isotope labeling and targeted proteomics approaches to identify trace bioactive components within PLPC-DB.
      The integration of these enrichment methodologies ensures that PLPC-DB maintains the highest analytical precision, allowing for accurate molecular characterization across research, diagnostic, and biopharmaceutical applications.

Composition and Functional Components of PLPC-DB The purified PLPC-DB composition is entirely free from animal or plant-derived materials, ensuring its compatibility with diverse biological systems while eliminating risks of genetic variability and immunological cross-reactivity. The formulation includes:

### Key Phospholipids

Phosphatidylcholine (PC)—Essential for cell membrane stability and lipid-mediated signaling.
Phosphatidylserine (PS)—Supports apoptosis regulation and immune response modulation.

### Bioactive Proteins

Integrins—Regulate cell adhesion and extracellular matrix interactions.
Tetraspanins—Facilitate cell migration, proliferation, and differentiation.

### Intercellular Signaling Molecules

Play a critical role in cellular communication, immune modulation, and regenerative processes.
Support biomolecular interactions necessary for homeostasis and tissue repair.
   The high purity and precision of PLPC-DB's biomolecular formulation enable its application in high-sensitivity research, molecular diagnostics, and regenerative therapies.

### Multidisciplinary Research Applications of PLPC-DB-

1. Biomedical and Molecular Research: PLPC-DB serves as a biotechnological resource for studying cellular processes, including:
   Tissue regeneration and repair mechanisms.
   Immune modulation studies and precision immunotherapy.
   Lipid metabolism and phospholipid-mediated intracellular signaling.
2. Veterinary and Aquaculture Biotechnology
   Allows researchers to investigate cellular and molecular interactions in animal systems without the risk of biological incompatibility.
   Supports research on veterinary immunotherapy, metabolic disease, and regenerative therapies.
   Enables the development of novel biomolecular interventions for animal health applications.

APPLICATIONS IN PLANT AND MICROBIOLOGICAL BIOTECHNOLOY: In addition to its biomedical and veterinary applications, PLPC-DB is a valuable tool for research in plant and microbial biotechnology, supporting studies on:

### Plant Biotechnology

Investigation of intercellular communication networks in plants.
Genetic enhancement strategies through phospholipid-mediated signal modulation.
Optimization of plant disease resistance mechanisms based on biomolecular interventions.

### Microbial Biotechnology

Research on microbiome-driven metabolic processes.
Examination of intercellular signaling pathways in microbial systems.
Enhancement of bacterial and fungal metabolic pathways for industrial biotechnology applications.
   The broad applicability of PLPC-DB extends beyond mammalian systems, enabling its use in microbiota research, plant biotechnology, and industrial bioengineering.

### Patentability, Innovation, and Scientific Impact of PLPC-DB

Contribution to Biotechnology and Research Advancements: PLPC-DB is designed to ensure high-standard biomolecular research in human therapeutic sciences, veterinary biotechnology, and aquaculture. By providing a high-purity, reproducible, and scalable phospholipoproteomic composition, it eliminates biological variability risks while complying with GRAS safety regulations. This guarantees a reliable, standardized input for scientific studies, optimizing the understanding of molecular interactions in complex biological systems and facilitating the development of new applications in biotechnology and translational medicine.

The makes it indispensable for advanced studies and gives it a clear patentability meritThe patentability of PLPC-DB lies in its unique, high-efficiency production process, which enables the manufacture of a phospholipoproteomic composition with purity levels exceeding 99%. This innovation overcomes the limitations of conventional bioinput sources, eliminating the regulatory and ethical constraints associated with animal- or plant-derived biomolecules.

### PLPC-DB has Broad Applicability Across Multiple Scientific Disciplines, Including:

Human biomedical research—Supporting molecular biology, regenerative medicine, and immune modulation studies.
Veterinary and aquaculture biotechnology—Providing a standardized, biosecure input for cellular and metabolic investigations in animal models.
Plant and microbiological research—Enabling the study of intercellular communication in plant and microbial ecosystems.
Theranostics and precision medicine—Facilitating biomolecular profiling, diagnostic advancements, and personalized therapeutic interventions.
   The ability of PLPC-DB to provide essential biomolecules with high purity, exceptional scalability, and reproducibility makes it a critical innovation in modern biotechnology, reinforcing its strong patentability standing, LaMext-generation scientific research, diagnostic applications, and translational medicine.

Core Innovations and Technological Advancements: PLPC-DB introduces multiple groundbreaking features that differentiate it from existing bioinput technologies while positioning it as a pioneering advancement in biomolecular research and applied biotechnology. These innovations significantly enhance biomolecular research, diagnostic methodologies, and the development of advanced therapeutic platforms.

This invention brings forward the following distinctive features, setting it apart from existing technologies:
A novel, high-precision purification process based on multi-stage adaptive ultrafiltration and centrifugation methodologies.
A fully optimized phospholipoproteomic composition, ensuring high bioactivity retention and exceptional batch-to-batch reproducibility.
A biocompatible formulation free from animal and plant-derived components, eliminating the risks of immunogenic contamination and cross-reactivity.
Scalability for research, diagnostic, and therapeutic applications, making it a cornerstone for high-sensitivity molecular investigations.
   These innovations place PLPC-DB at the forefront of biomolecular research, reinforcing its scientific merit and patentable uniqueness.

### Innovative Purification Process:

Differentiation from Existing Technologies
PLPC-DB is manufactured using a highly optimized, multi-stage purification system, which integrates:
High-speed centrifugation (>10,000 RPM)—Enables precise molecular fractionation and effective removal of non-essential biomolecules.
Adaptive ultrafiltration with tunable membrane porosity (>3 kDa)—Ensures selective retention of bioactive proteins while eliminating contaminants.
Adjustable process parameters—Customizable centrifugation speed, cycle number, and duration, ensuring optimized purity and functional consistency.

This flexible and adaptive purification methodology preserves bioactive integrity, making PLPC-DB an exceptional tool for scientific and clinical applications, a feature not achieved by conventional bioinput purification techniques.

### (b) Patentability Justification

The exclusive nature of PLPC-DB's purification methodology strengthens its patentability argument. The process is:
Customizable and adaptive, allowing precise modifications in centrifugation and ultrafiltration settings to meet specific research and therapeutic objectives.
Scientifically standardized, ensuring consistent biomolecular composition across diverse applications and laboratory settings.
Designed to retain structural integrity, preserving the functionality of phospholipids, proteins, and peptides across a range of experimental conditions.

The ability to generate a phospholipoproteomic composition with >99% purity while maintaining exceptional reproducibility places this invention beyond the capabilities of existing technologies, reinforcing its patentable uniqueness and market differentiation.

Comparison with Conventional Bioinput Technologies
PLPC-DB represents a significant advancement in the standardization of bioinputs, surpassing traditional purification methods in:
Purity and Reproducibility—Achieving higher biomolecular integrity and functional stability than static, fixed-parameter purification systems.
Process Adaptability—Allowing precise control over bioactive retention, enabling its use across various biotechnological applications.
Biological Compatibility—Providing a contamination-free, reproducible alternative to traditional animal- and plant-derived bioinputs.

This technological superiority establishes PLPC-DB as the first fully standardized, ultrapure, and functionally stable phospholipoproteomic composition available for biomolecular research, diagnostics, and next-generation therapies.

### Validation of Market Differentiation and Research Reproducibility

To support its patentability and commercial viability, comparative studies have been conducted, demonstrating that PLPC-DB:
Exhibits higher batch-to-batch consistency, reducing variability in biomolecular composition.
Maintains superior purity levels, preventing degradation and maintaining bioactivity over time.
Outperforms conventional biomolecular purification techniques, enabling greater functional stability and molecular integrity.
   These scientific and industrial validations establish PLPC-DB as an unmatched innovation in the field of biotechnology, reinforcing its strong patentability standing and research applicability.

### Scientific and Commercial Relevance of PLPC-DB

The scientific impact and commercial potential of PLPC-DB extend across multiple sectors, including:
Precision medicine and personalized therapeutic development.
Advanced molecular diagnostics and biomarker-driven research.
Regenerative medicine and cell-based therapeutic engineering.
Veterinary and aquaculture biotechnology for non-animal-derived molecular studies.
Plant and microbial biotechnologies for genetic optimization and metabolic profiling.
   The combination of groundbreaking purification technologies, reproducible molecular composition, and scalable production positions PLPC-DB as a leading innovation in biomolecular science and applied research.
   By delivering a high-purity, reproducible, and functionally stable phospholipoproteomic composition, PLPC-DB sets a new benchmark for high-sensitivity scientific applications, reinforcing its patentability merit and long-term value in research and industry.

### Scientific Validation, Commercial Positioning, and Regulatory Alignment of PLPC-DB

Purified Composition Without Direct Cellular Manipulation

(a) Differentiation from Other Bioinputs
Unlike biological inputs that rely on direct cellular manipulation, PLPC-DB is produced without any form of genetic modification or cellular engineering. This distinction offers several advantages:
Elimination of biological variability, ensuring that each batch maintains consistent molecular composition.
Reduced contamination risk, minimizing potential immunogenic and xenobiotic residues.
Increased research reliability, making it an ideal input for regulatory-compliant biomedical studies.

### (b) Regulatory and Commercial Relevance

By eliminating cellular manipulation, PLPC-DB simplifies regulatory pathways, facilitating its commercialization in diverse scientific and clinical markets. This advantage enhances:
Compliance with global safety and ethical research guidelines, making it widely acceptable across biopharmaceutical and research institutions.
Scalability for high-throughput applications, allowing its integration into large-scale production pipelines without additional regulatory constraints.

PLPC-DB achieves a purity level exceeding 99%, preserving the functional bioactivity of phospholipids, proteins, and peptides, including:
Phosphatidylserine (0.5-2% of total weight)—Essential for cellular apoptosis regulation and immune signaling.
Phosphatidylcholine (1-5%)—A fundamental component of membrane integrity and lipid metabolism.
Phosphatidylethanolamine (0.1-0.5%)—Supports cell membrane flexibility and protein-lipid interactions.
Bioactive proteins, such as integrins and tetraspanins, critical for cellular adhesion, migration, and extracellular matrix interactions.
Signaling peptides, optimizing biochemical communication between cells and enhancing regenerative processes.

The structural stability and bioactivity of PLPC-DB make it an indispensable resource for cell signaling studies, regenerative medicine, and advanced molecular diagnostics.

Versatility of Applications and Market Expansion Potential

### (a) Broad Research and Clinical Applications

PLPC-DB is designed for a wide range of scientific and medical applications, covering:
Molecular biology and proteomics—Used in advanced studies of lipid metabolism, biomarker discovery, and cell regeneration.
Immune support protocols and cellular modulation—Applied in personalized medicine, immunotherapy, and metabolic health research.
Therapeutic biomaterials development—Supports cell-based therapies, scaffold integration, and tissue engineering.

### (b) Market Impact and Growth Potential

PLPC-DB's multidisciplinary adaptability makes it highly attractive to various research and clinical disciplines, including:
Preclinical and clinical trials, ensuring standardized input for biomolecular investigations.
Applied biotechnology, integrating high-purity bioinputs into diagnostic and therapeutic innovations.
Innovative health products, supporting the development of next-generation biomedical solutions.
   Its ability to address evolving market demands in precision medicine and regenerative therapies reinforces its commercial viability and strategic relevance in the biotechnology sector.

### Scientific Validation and Research-Based Evidence

### (a) Differentiation Through Scientific Support

PLPC-DB is backed by rigorous scientific studies, demonstrating its efficacy and safety across multiple biomedical applications. These studies provide ** key validation in:
Preclinical and translational research, proving its effectiveness in cell regeneration and immune response modulation.
Mass spectrometry-based biomolecular analysis, confirming its ultrapure phospholipid-protein composition.
Comparative studies with existing bioinputs, highlighting its superior functional stability and batch-to-batch reproducibility.

### (b) Strength in Clinical and Regulatory Approvals

The inclusion of preclinical and clinical data within the patent documentation strengthens confidence in PLPC-DB's biomedical applications, facilitating:
Faster regulatory approval processes due to its validated safety profile.
Wider acceptance in pharmaceutical and diagnostic research, ensuring efficient integration into high-impact medical studies.
   This scientific robustness promotes PLPC-DB's rapid adoption in global research initiatives.

### Standardized and Scalable Production Process

(a) High-Volume Production without Compromising Quality
PLPC-DB is produced under a standardized, scalable manufacturing process, ensuring:
Consistent purity and molecular integrity, meeting the highest regulatory and research standards.
Flexible adaptation to industrial-scale production, making it suitable for both small-scale laboratories and large-scale biomanufacturing.
Optimization for diverse biomolecular applications, allowing customization of formulations based on research needs.

### (b) Strategic Market Competitiveness

The scalability of PLPC-DB's production process provides:
A competitive edge in global biotechnology markets, making it an attractive choice for research institutions and biopharmaceutical developers.
A reliable input for multicenter studies, ensuring global reproducibility in biomolecular investigations.
   PLPC-DB's manufacturing adaptability and high-purity standard reinforce its position as a leading bioinput for next-generation scientific and clinical applications.
   Alignment with Health and Wellness Market Trends
   (a) Safe and Effective Biotechnology Solutions: The growing emphasis on safe, effective, and ethically sourced bioinputs underscores the relevance of PLPC-DB in the expanding health and wellness industry.
Immune support and metabolic health research-Supporting therapeutic innovations targeting chronic inflammation and autoimmune diseases.
Precision biotechnology solutions-Providing highly purified molecular inputs tailored for therapeutic applications.
Nutraceutical and functional biomolecule development-Contributing to next-generation health optimization strategies.
   (b) Commercial Potential and Expansion Opportunities: By aligning with current advancements in biotechnology and personalized medicine, PLPC-DB enhances:
Market attractiveness to pharmaceutical and research institutions, ensuring broad commercialization potential.
Cross-disciplinary applications, making it valuable for diagnostics, drug delivery, and therapeutic development.
   Its market positioning as a next-generation bioinput enhances its commercial success and long-term biomedical impact.

Regulatory Compliance and Global Market Accessibility: PLPC-DB is an ultrapurified protein-based composition developed for biomedical research, diagnostics, and molecular biotechnology. It achieves purity levels exceeding 99% through a highly controlled, standardized purification process, ensuring:

### Regulatory Compliance and Global Market Accessibility

PLPC-DB is an ultrapurified protein-based composition developed for biomedical research, diagnostics, and molecular biotechnology. It achieves purity levels exceeding 99% through a highly controlled, standardized purification process, ensuring:
Elimination of biological contaminants and unwanted molecular variability.
Compliance with international safety regulations, supporting multicenter research and biopharmaceutical development.

### Regulatory Certifications and Global Compliance

PLPC-DB fully adheres to recognized international safety standards, facilitating its approval for research and industrial use:
GRAS (Generally Recognized as Safe) by the U.S. FDA-Ensuring safe inclusion in regulated biomedical studies.
Novel Food (European Union) certification-Supporting biotechnological innovation and product safety within the EU market.
NHP (Natural Health Products, Canada)—Allowing research integration into North American pharmaceutical and healthcare industries.
   These regulatory credentials reinforce PLPC-DB's position as a globally accepted, standardized bioinput, providing a safe, reproducible, and functionally optimized biomolecular tool for scientific research and therapeutic advancements.

### CONCLUSION

PLPC-DB represents a significant advancement in biomolecular standardization, functional stability, and high-purity research applications, ensuring:
High reproducibility and batch-to-batch molecular consistency.
Cross-disciplinary applicability in biomedical, pharmaceutical, and diagnostic fields.
Full compliance with international safety and regulatory standards.
Market differentiation through its patent-protected, high-purity purification methodology.

### High-Purity, Regulatory-Compliant, and Functionally Optimized Biomolecular Input: PLPC-DB

Addressing the Need for High-Purity, Non-Animal/Non-Plant Biomolecular Inputs: PLPC-DB has been developed to meet the urgent demand for ultrapure bioinputs that are free from animal or plant-derived materials, thereby eliminating:
Biological variability, which is inherent in natural-source biomolecules and affects reproducibility.
Cross-reactivity risks, ensuring consistency in molecular interactions and analytical outcomes.
Regulatory and ethical constraints, providing a fully compliant, standardized alternative for global research applications.
   Through a highly controlled purification process, PLPC-DB achieves a purity level exceeding 99%, making it a reliable bioinput for advanced studies in:
Cell signaling—Supporting research into intracellular communication and biochemical cascades.
Tissue differentiation—Providing a high-fidelity model for cellular specialization studies.
Tissue regeneration—Enhancing understanding of phospholipid-mediated repair mechanisms.
Bioactive protein and phospholipid—based diagnostic technologies-Facilitating precision biomarker development and molecular detection platforms.
   The scientific precision and batch-to-batch reproducibility of PLPC-DB ensure its suitability for large-scale, high-impact biomedical research and industrial applications.

A Fully Standardized and Internationally Compliant Bioinput: This invention delivers an ultrapurified phospholipoproteomic composition, engineered to meet the highest global standards for safety, purity, and functional stability. PLPC-DB is highly compatible with:
Molecular diagnostics, enabling high-sensitivity biomarker detection and disease profiling.
Immunomodulation research, supporting immune cell regulation and targeted therapy studies.
Metabolic support applications, enhancing precision-targeted lipid metabolism research.
   By providing a reproducible and scalable bioinput, PLPC-DB facilitates:
Advanced scientific research, ensuring reliable data across multicenter studies. Biotechnological innovation, enabling breakthroughs in protein-based and lipid-mediated molecular therapeutics.
Translational medicine, bridging basic molecular insights with applied therapeutic advancements.
   Its non-animal, non-plant-derived nature positions PLPC-DB as a pioneering solution for global life sciences research and next-generation diagnostic development.

Innovative Purification Process and Molecular Optimization: PLPC-DB is a next-generation phospholipoproteomic composition, obtained from cell culture supernatants using a multi-stage purification system that integrates:
High-speed centrifugation (>10,000 RPM)—Ensuring precise molecular fractionation and bioactive retention.
Ultrafiltration with adjustable porosity membranes (minimum cut-off: 3 kDa)—Allowing:
   Selective retention of bioactive proteins and phospholipids.
   Exclusion of non-target molecules and contaminants.
   Optimization of bioactive molecule integrity and stability.
      This purification strategy ensures that PLPC-DB retains its full functional bioactivity, including:
Essential phospholipids—Phosphatidylserine, phosphatidylcholine, phosphatidylethanolamine, which regulate membrane stability, apoptosis, and cellular repair.
Specific bioactive proteins—Integrins and tetraspanins, critical for cellular adhesion, immune modulation, and extracellular matrix interactions.
Signaling peptides—Mediating intercellular communication and supporting tissue regeneration.
   The process parameters (centrifugation speed, cycle number, membrane porosity selection) are fully adjustable, enabling precise molecular separation and customization for diverse biomedical and industrial applications.
   This adaptability guarantees that PLPC-DB consistently meets the highest benchmarks for quality, reproducibility, and biofunctional stability, making it a cornerstone bioinput for biomolecular research and diagnostic applications.

Compliance with International Regulations and Multicenter Research Integration: PLPC-DB has been developed in strict compliance with global regulatory frameworks, ensuring its safe and effective use in international research collaborations.

### It Adheres to:

GRAS (Generally Recognized as Safe) by the U.S. FDA—Ensuring regulatory acceptance for biomedical research applications.
Novel Food Regulation by the European Union—Supporting its use in biotechnological innovations and nutraceutical formulations.
NHP (Natural Health Products) Regulation by Canada—Facilitating its integration into therapeutic research and clinical trials.
   Key regulatory advantages of PLPC-DB include:
Minimized biological variability, improving experimental reproducibility in multicenter studies.
No immunogenic or cross-reactive risks, ensuring biocompatibility across human, veterinary, and microbiological applications.
Optimized purity and stability, meeting stringent biopharmaceutical and diagnostic quality standards.
   By removing regulatory barriers associated with conventional bioinputs, PLPC-DB is positioned as a leading bioinput for precision research, molecular diagnostics, and next-generation biotherapeutic advancements.
   Its high standardization, scalability, and global regulatory alignment ensure its adoption in biomedical research, pharmaceutical innovation, and cutting-edge diagnostic development.

### Differentiation, Compliance, and Scientific Impact of PLPC-DB

PLPC-DB stands apart from other protein-based biomolecular inputs due to its highly controlled purification process, international regulatory compliance, and broad scientific applicability. Its unique purity, stability, and biocompatibility ensure optimal performance across biomedical, diagnostic, and therapeutic research settings.

### Key Differentiating Features of PLPC-DB

### 1. Standardized Compatibility and Safety

PLPC-DB is developed under a strictly controlled protocol that eliminates the need for animal or plant-derived components, reducing the risks associated with:
Biological variability, ensuring a consistent molecular profile in all batches.
Cross-reactivity, avoiding unwanted immune responses or unpredictable biochemical interactions.
External contaminants, ensuring that each sample remains free from residual xenobiotic materials.
   This results in a fully standardized, highly reproducible bioinput, optimized for multicenter studies and global scientific research applications.

### 2. Optimization of the Purification Process

PLPC-DB undergoes a multi-stage purification protocol, integrating:
High-speed centrifugation—Facilitating effective molecular fractionation while preserving structural integrity.
Precision ultrafiltration—Selectively retaining bioactive proteins and phospholipids while eliminating cellular debris.
Molecular stabilizers—Preventing the loss of bioactive functionality throughout the purification process, ensuring maximum bioavailability and stability.
   This process guarantees exceptional biomolecular stability, allowing PLPC-DB to retain its high-purity phospholipid-protein composition over extended periods of research and application.
   3. Compliance with International Regulations
   PLPC-DB meets and exceeds globally recognized biomolecular safety and regulatory standards, ensuring seamless acceptance across multiple jurisdictions:
GRAS (Generally Recognized as Safe)—U.S. FDA
   Guarantees safety and compliance for biomedical and pharmaceutical research applications.
Novel Foods Regulation—European Union
   Allows biotechnological integration within European regulatory frameworks, ensuring acceptance in molecular research and clinical trials.
NHP (Natural Health Products) Regulation—Canada
   Certifies biocompatibility and molecular reproducibility, facilitating multicenter research and therapeutic studies.
      The regulatory adherence of PLPC-DB simplifies its adoption in advanced scientific and diagnostic applications, providing researchers and developers with a reliable, compliant, and reproducible biomolecular input.

### 4. Multidisciplinary Applications Across Scientific and Medical Fields

PLPC-DB is a highly versatile bioinput, used in:
Biomedical Research—Serving as a high-purity molecular input for proteomics, lipidomics, and cell biology.
Biomarker Development—Optimizing the detection and validation of disease-specific molecular signatures.
Diagnostic Technologies—Supporting high-precision diagnostic assays such as ELISA, immunofluorescence, and mass spectrometry.
Tissue Regeneration Studies—Facilitating wound healing, extracellular matrix remodeling, and cell differentiation research.
Immunomodulation Protocols—Providing an optimized biomolecular platform for immune response modulation and therapeutic interventions.
   With a purity level exceeding 99%, PLPC-DB is ideal for cutting-edge molecular investigations, ensuring superior performance in biomedical and translational research settings.

Patent Strength and Global Market Relevance: PLPC-DB's unique composition and regulatory compliance differentiate it from existing protein-based bioinputs, reinforcing its intellectual property protection and market viability.
Strengthened Patent Claims—PLPC-DB's innovative purification methodology, non-animal-derived composition, and scalable production process justify its patentability as a pioneering molecular input.
Regulatory Compliance and Market Access—Its alignment with international safety and purity standards ensures acceptance in global scientific and biopharmaceutical markets.
Multicenter Research Integration—Its reproducibility across research institutions and commercial applications increases its credibility and demand within the biotechnology sector.
   The clarity and strength of these advantages position PLPC-DB as a fundamental bioinput for next-generation biomedical and diagnostic applications.

### Materials and Equipment:

### Standardized Production Protocol for High-Purity Phospholipoproteomic Composition

The primary objective of PLPC-DB's production protocol is to obtain a phospholipoproteomic composition with a minimum purity of 99%, ensuring:
Stability of bioactive molecules, preserving their structural and functional integrity.
Consistency across all production batches, ensuring reproducibility in scientific and therapeutic applications.
Long-term safety and viability, allowing seamless integration into regulatory-compliant research settings.
   This protocol has been designed to meet GRAS standards, ensuring the highest level of biomolecular safety and functionality in advanced research and diagnostic applications.

### Standardized Production Process and Scientific Applications of PLPC-DB

Production Process and Quality Control Standards: The PLPC-DB production protocol follows a rigorously controlled, multi-step purification and validation system, ensuring purity, stability, and functional integrity across all production batches.

### Key Quality Control Stages:

1. Purity Verification via HPLC and Mass Spectrometry
   Phospholipid and bioactive protein composition is analyzed using High-Performance Liquid Chromatography (HPLC) and Mass Spectrometry to confirm that the formulation meets the >99% purity standard.
   Detection of contaminants and process residuals is performed, ensuring that each batch is free from unwanted biological or chemical impurities.
2. Functionality Assessment for Bioactive Molecules
   Specific biological assays are conducted to validate the ability of bioactive proteins and phospholipids to:
      Modulate cell signaling pathways related to cellular adhesion, immune response, and metabolic regulation.
      Enhance intercellular communication, ensuring optimal functionality in tissue engineering and immunomodulation studies.
3. Stability Testing for Long-Term Bioactivity
   Accelerated stability studies are performed under controlled environmental conditions to confirm:
      Structural and biochemical integrity of the formulation over prolonged storage.
      Retention of bioactivity in various storage conditions, ensuring usability in real-world research and diagnostic applications.
4. Documentation and Process Traceability
   Complete traceability of every production stage, ensuring compliance with:
      Good Manufacturing Practices (GMP).
      GRAS regulatory guidelines (U.S. FDA).
      International quality standards for biomolecular inputs (ISO, EU Novel Foods, NHP Canada).
         This highly standardized production process ensures that PLPC-DB remains a reliable, high-purity bioinput for biomedical, biotechnological, and therapeutic applications, providing batch-to-batch consistency and global regulatory compliance.

Purified and Safe Input for Advanced Biomolecular Research: PLPC-DB is an Ultrapurified Phospholipoproteomic Compound, engineered to be a safe and highly compatible input for biomedical research, diagnostics, and applied biotechnology.

### Key Features of PLPC-DB:

Non-animal, non-plant origin, eliminating risks of biological variability and cross-reactivity.
Standardized purification process, ensuring batch consistency and high bioactivity retention.
GRAS-compliant molecular profile, ensuring safety in global research applications.
   By eliminating variability risks associated with conventional bioinputs, PLPC-DB improves reproducibility in multicenter studies, making it an optimal tool for clinical research, diagnostic applications, and next-generation biotechnology.

Multidisciplinary Applications of PLPC-DB in Biomedical Research: PLPC-DB serves as a core biomolecular input in multiple disciplines, offering a purified and highly stable source of phospholipids, proteins, and peptides. Its high purity and compatibility with regulatory standards ensure its safe integration into multicenter studies, biomarker research, and advanced diagnostic applications.

### 1. Cell Signaling and Tissue Differentiation Studies

PLPC-DB is an essential component for cellular signaling investigations, providing a precisely defined biomolecular composition that enables:
Activation of critical signaling pathways, including:
   PI3K/AKT—Regulating cell growth, differentiation, and survival.
   NF-κB—Controlling immune response and inflammation.
   MAPK/ERK—Modulating cell proliferation and tissue regeneration.
In vitro and in vivo applications, ensuring high reproducibility in stem cell differentiation and regenerative tissue models.
Optimization of bioactive molecule delivery, ensuring consistent biomolecular interactions in experimental settings.

By providing an ultrapure, standardized bioinput, PLPC-DB ensures precise, reproducible, and scalable research in regenerative medicine and cellular biology.

### 2. Biomarker Development and Immunomodulation Protocols

PLPC-DB serves as a highly stable, reproducible biomolecular platform for biomarker discovery and immune modulation research.
Supports the development of clinical biomarkers, enabling accurate detection of inflammation, cancer progression, and metabolic imbalances.
Enhances diagnostic precision, facilitating biomarker identification through:
   ELISA-based high—sensitivity assays.
   Mass spectrometry—driven proteomics and phospholipid analysis.
   Immunofluorescence—based immune cell profiling.
Customizable for immune support and cellular modulation protocols, ensuring:
   Improved precision in personalized therapeutic approaches.
   Standardized molecular targeting for diagnostic and treatment applications.
      PLPC-DB's high bioactivity and reproducibility make it an optimal input for immunology research, disease biomarker studies, and personalized medicine applications.

### 3. Research in Regeneration and Metabolic Support

PLPC-DB is fully compatible with preclinical models for tissue regeneration and metabolic research, providing:
A controlled biomolecular environment for:
   Stem cell differentiation into functional tissues.
   Wound healing and extracellular matrix remodeling studies.
   Organ and tissue regeneration in bioengineering applications.
Metabolic support research, optimizing studies on:
   Lipid metabolism and phospholipid-mediated cellular responses.
   Energy balance in tissue recovery models.
   Biochemical interactions in metabolic disorders and chronic disease studies.
      By ensuring high purity and functional consistency, PLPC-DB enables reliable, high-sensitivity research in regenerative biology and metabolic health applications.

Phospholipoproteomic Composition: High-Purity and Functional Integrity: PLPC-DB is a next-generation ultrapure phospholipoproteomic composition designed to meet the highest standards in biomedical research, molecular diagnostics, and therapeutic applications. The composition maintains a purity exceeding 99%, ensuring structural stability and functional bioactivity.

### Key Phospholipid Components and Their Functional Roles

PLPC-DB contains essential phospholipids that play a critical role in cell membrane stability, biochemical signaling, and metabolic regulation:
Phosphatidylserine (0.5-2.0% of total weight)—Supports apoptosis regulation, immune signaling, and neuroprotective functions.
Phosphatidylcholine (1.0-5.0% of total weight)—A primary component in membrane biogenesis, lipid metabolism, and neurotransmitter synthesis.
Phosphatidylethanolamine (0.1-0.5% of total weight)—Facilitates membrane curvature dynamics, protein anchoring, and intracellular vesicular transport.

### Advanced Purification Methodology

The ultrapure nature of PLPC-DB is achieved through a customized, high-precision purification process integrating:
High-speed centrifugation (>10,000 RPM)—Ensuring efficient molecular fractionation and contaminant removal.
Ultrafiltration with adaptable porosity membranes (>3 kDa cut-off limit)—Optimizing component separation while retaining bioactive integrity.
   This optimized purification strategy provides a composition highly suitable for advanced research in molecular biology, regenerative medicine, and cell signaling studies, ensuring:
Batch-to-batch reproducibility, crucial for multicenter scientific investigations.
Retention of bioactive functionality, making it ideal for high-sensitivity analytical assays and diagnostic technologies.
Biocompatibility with therapeutic applications, facilitating its use in metabolic and immune-modulation studies.

Purified Bioactive Proteins: Essential Regulators of Cellular Function: PLPC-DB contains a well-defined fraction of purified bioactive proteins, which play a key role in cellular communication and biochemical regulation. These proteins are stringently purified and validated through functional assays to maintain their structural integrity and biological efficacy.
Integrins (0.5-1.5%)—Regulate cell adhesion, migration, and extracellular matrix interactions.
Tetraspanins (0.5-1.5%)—Play a role in cellular differentiation, immune response coordination, and intracellular signaling.
   These proteins enhance intercellular interactions, ensuring high-precision research applications in immunology, oncology, and tissue engineering.

Cell Signaling and Communication Peptide: PLPC-DB incorporates a fraction of purified peptides (0.2-1.0%) specifically selected to enhance intercellular communication and optimize biochemical signal transduction.
These peptides regulate signal cascades in cellular regeneration and immune modulation.
They support tissue engineering applications by enhancing cellular differentiation and metabolic communication.
Their stability and bioactivity are confirmed by analytical assays and cell viability studies.
   These components reinforce PLPC-DB's utility in research on molecular signal transduction and targeted therapeutic development.

### Illustration of the Phospholipoproteomic Purification Process

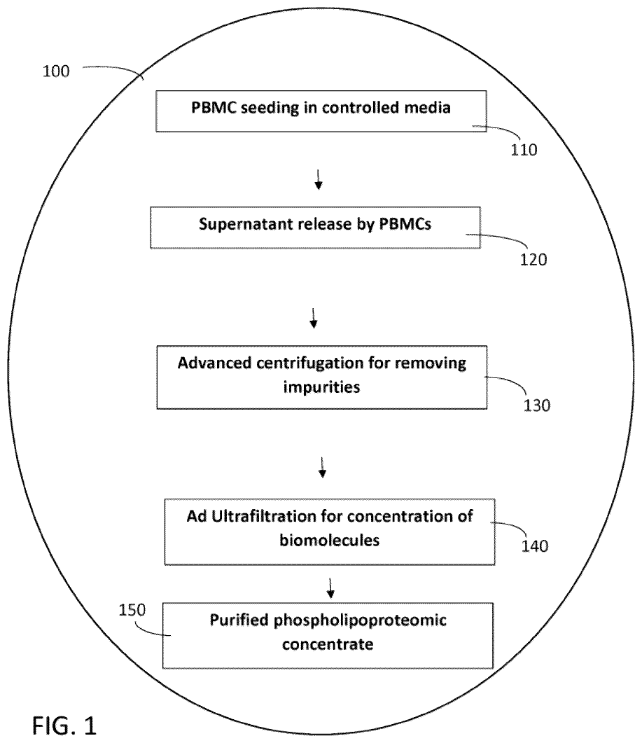

FIG. 1 is a flowchart detailing the sequential steps involved in the purification process of phospholipoproteomic compositions for biotechnology, molecular diagnostics, and therapeutic applications. This figure provides a graphical representation of the high-speed centrifugation and ultrafiltration methodology, illustrating how bioactive phospholipids, proteins, and peptides are selectively retained while contaminants and non-essential components are eliminated. The process for obtaining purified phospholipoproteomic compositionsis a standardized and scalable method for the purification of phospholipoproteomic compositions, optimized for biotechnological applications, molecular research, and diagnostic studies. The process flow 100 consists of sequential steps designed to ensure high-purity isolation of phospholipids, bioactive proteins, and peptides. Eliminate non-essential cellular components and contaminants, retain bioactive functionality and molecular stability for advanced research and industrial applications. The following steps outline the highly controlled purification methodology used to obtain PLPC-DB, ensuring batch-to-batch reproducibility and compliance with regulatory standards.

Step 110: Seeding of Cells in Controlled Medium: The process begins with the seeding of selected cell lines, such as Peripheral Blood Mononuclear Cells (PBMCs), in a strictly controlled culture environment. The medium composition and incubation conditions are optimized to maximize cell viability and bioactive molecule secretion, enhance the production of phospholipids, proteins, and intercellular signaling molecules and ensure controlled metabolic conditions, eliminating unwanted cellular stress responses that could affect the final bioactive composition. This carefully regulated seeding process ensures that the supernatant collected in subsequent steps contains high concentrations of target biomolecules, making it suitable for puStep 120: Supernatant Collection from Cultured Cells

Once the cultured cells have reached optimal bioactive molecule production, the next step involves the collection of the supernatant—the extracellular fluid containing target phospholipids, proteins, and peptides. The supernatant is carefully extracted under sterile conditions to prevent contamination and degradation and immediately processed to retain biomolecular stability, preventing unwanted enzymatic degradation. The supernatant is subjected to preliminary filtering, ensuring that large cellular debris and non-target biomolecules are efficiently removed. This step ensures that only bioactive molecules of interest proceed to the advanced purification stages, maintaining the functional integrity of phospholipids and proteins in the final product.

Step 120: Supernatant Collection from Cultured Cells: Once the cultured cells have reached optimal bioactive molecule production, the next step involves the collection of the supernatant—the extracellular fluid containing target phospholipids, proteins, and peptides.

The supernatant is:
Carefully extracted under sterile conditions to prevent contamination and degradation.
Immediately processed to retain biomolecular stability, preventing unwanted enzymatic degradation.
Subjected to preliminary filtering, ensuring that large cellular debris and non-target biomolecules are efficiently removed.
   This step ensures that only bioactive molecules of interest proceed to the advanced purification stages, maintaining the functional integrity of phospholipids and proteins in the final product.

### Step 130: Advanced Centrifugation for Impurity Removal

The collected supernatant is subjected to a high-speed centrifugation process, ensuring efficient removal of unwanted cellular contaminants and impurities.
Centrifugation parameters:
   Speeds exceeding 10,000 RPM, allowing for precise biomolecular separation based on density and molecular properties.
   Customizable cycle durations, ensuring maximum retention of bioactive proteins, phospholipids, and peptides.
   Optimization based on sample characteristics, preventing structural degradation or loss of functional molecules.

Step 140: Ultrafiltration for Biomolecular Concentration: Following centrifugation, the phospholipoproteomic fraction undergoes ultrafiltration, ensuring:
Selective retention of bioactive phospholipids, proteins, and peptides, while removing low-molecular-weight contaminants.
Optimization of molecular separation using customizable membrane porosity, ranging from 3 kDa to 50 kDa.
Final enrichment of the purified bioactive fraction, making it suitable for biomedical research, diagnostics, and therapeutic applications.
   By applying adaptive ultrafiltration techniques, the PLPC-DB composition retains its functional stability, reproducibility, and biocompatibility, ensuring it meets scientific and industrial standards for molecular research and diagnostics.

Step 150: Final Purification and Phospholipoproteomic Concentrate: The final stage of the purification process results in the ultrapure phospholipoproteomic concentrate, containing:
Purified phospholipids, including phosphatidylserine, phosphatidylcholine, and phosphatidylethanolamine.
Bioactive proteins, such as integrins and tetraspanins, crucial for cellular communication and extracellular matrix interactions.
Signaling peptides, facilitating biochemical processes related to immune modulation and tissue regeneration.

### This Final Formulation is:

Validated for purity and functionality, ensuring biochemical stability under research and therapeutic conditions.
Batch-tested for reproducibility, maintaining consistency in large-scale biomolecular applications.
Compliant with international safety and purity standards, enabling global research integration.
   The resulting PLPC-DB composition is an advanced, scientifically optimized biomolecular input, suitable for precision research, next-generation diagnostics, and personalized medicine applications.

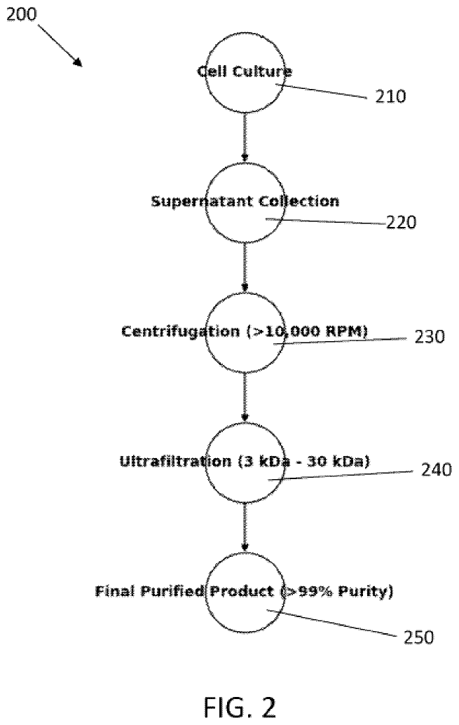

FIG. 2 is a diagram 200 showing the PLPC-DB purification workflow including a multi-stage process for ultrapure biomolecular composition. The purification of PLPC-DB follows a multi-step methodology combining high-speed centrifugation and selective ultrafiltration, ensuring the removal of contaminants and the retention of bioactive phospholipids, proteins, and peptides. This process achieves a final purity exceeding 99%, maintaining biomolecular stability for biomedical applications.

### Process Explanation:

Cell Culture 210—Initial phase where PBMCs are grown in controlled conditions.
Supernatant Collection 220—Extraction of the medium containing bioactive molecules.
Centrifugation 230—High-speed separation (>10,000 RPM) of non-essential components and impurities.
Ultrafiltration 240—Removal of contaminants and size-based molecular separation (3-50 kDa).
Final Purified Product 250—Highly stable PLPC-DB with a purity level exceeding 99%.

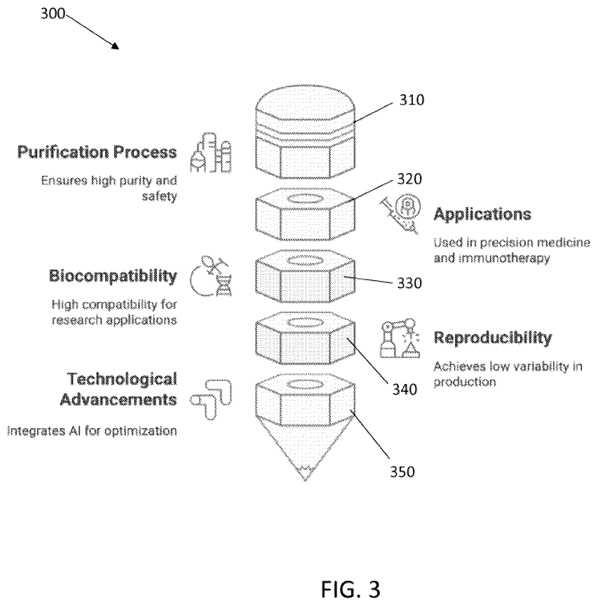

FIG. 3 is a diagram 300 showing components and applications of the PLPC-DB. PLPC-DB is designed as a highly purified and biocompatible phospholipoproteomic composition, ensuring superior safety, stability, and functional integrity for advanced biomedical applications. This diagram illustrates the core structural elements that define PLPC-DB, including its optimized purification process, biocompatibility, and AI-driven technological advancements. Its high reproducibility and compliance with international safety standards make it a critical tool for precision medicine, immunotherapy, and molecular research.
Breakdown of Key Components in PLPC-DB Composition:
The purification process 310 ensures high purity and safety through a multi-step purification system.
Eliminates biological contaminants while preserving bioactive integrity.
Biocompatibility 330 for high compatibility in research and therapeutic applications and eliminates biological variability by avoiding animal- and plant-derived components.
Technological Advancements 350 integrate AI-driven optimization for enhanced molecular retention and selectivity and allows real-time process adjustments to improve reproducibility and consistency.
Reproducibility 340 in the composition achieves low variability in production, ensuring batch-to-batch consistency (<2%) and provides scalability for clinical and research applications.
Applications 320 are used in precision medicine, immunotherapy, and regenerative medicine and facilitate targeted research in molecular diagnostics and cellular therapies.

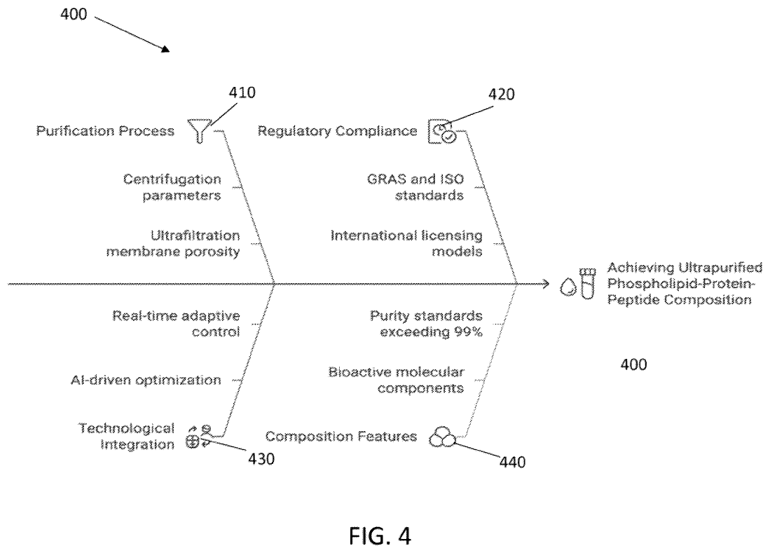

FIG. 4 is a chart 400 of overcoming purification challenges with PLPC-DB. PLPC-DB achieves a result 450 of an ultrapure phospholipid-protein-peptide composition by integrating advanced purification techniques, real-time adaptive control, and regulatory compliance. By optimizing centrifugation parameters, ultrafiltration membrane porosity, and AI-driven process improvements, PLPC-DB ensures a purity standard exceeding 99%, making it an ideal bioinput for precision medicine, immunotherapy, and molecular research. FIG. 4 includes:
The purification process 410 is optimized centrifugation parameters for effective separation. The ultrafiltration membrane porosity is adjusted to retain bioactive molecules while eliminating contaminants.
For regulatory compliance 420, GRAS and ISO standards ensure global acceptance and safety aand international licensing models facilitate research and commercialization worldwide.
Technological Integration 430 includes real-time adaptive control which optimizes purification in response to live biomolecular data. AI-driven optimization enhances reproducibility and efficiency in purification.
Composition Features 440 include purity standards exceeding 99%, ensuring high molecular integrity and bioactive molecular components are retained for optimal research and therapeutic applications.

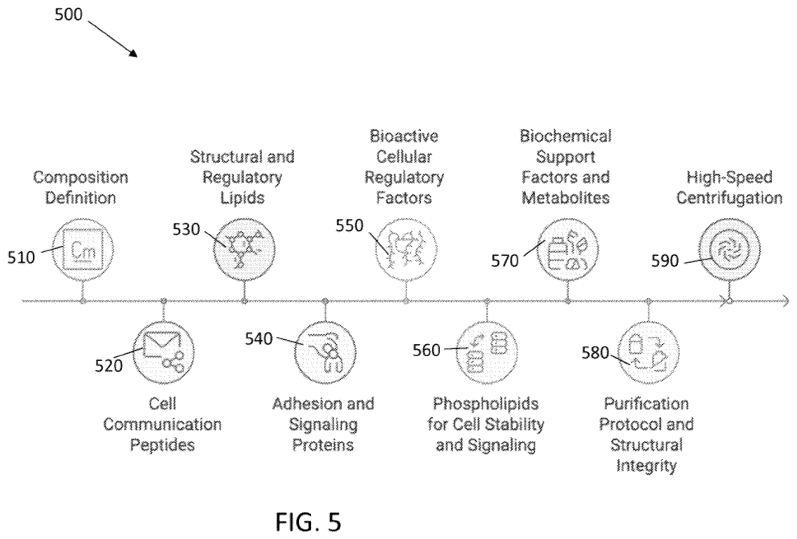

FIG. 5 is a diagram 500 showing the purification process of the phospholipoproteomic composition. The purification of the phospholipoproteomic composition follows a structured process designed to achieve optimal molecular integrity, functional stability, and regulatory compliance. This workflow ensures the selective retention of essential biomolecules, including structural phospholipids, bioactive proteins, and cellular regulatory factors, while maintaining the highest purity and reproducibility standards. By integrating high-speed centrifugation with advanced molecular filtration techniques, this purification method enables the development of high-precision bioinputs for biomedical and biotechnological applications.
Breakdown of Key Stages in the Purification Process:
   Composition definition 510 including establishing molecular criteria for phospholipids, proteins, and peptides.
   Cell communication peptides 520 essential for intercellular signaling and biochemical transmission.
   Structural and regulatory lipids 530 contributing to membrane stability and intracellular processes.
   Adhesion and signaling proteins 540 supporting cellular interactions and extracellular matrix integrity.
   Bioactive cellular regulatory factors 550 modulating immune response and cellular differentiation.
   Phospholipids for cell stability and signaling 560 ensuring membrane integrity and metabolic efficiency.
   Biochemical support factors 570 and metabolites providing essential molecular interactions for cell function.
   Purification protocol and structural integrity 580 ensuring biomolecular stability throughout the process.
   High-speed centrifugation 590 facilitating the separation of biomolecules for optimal purity.

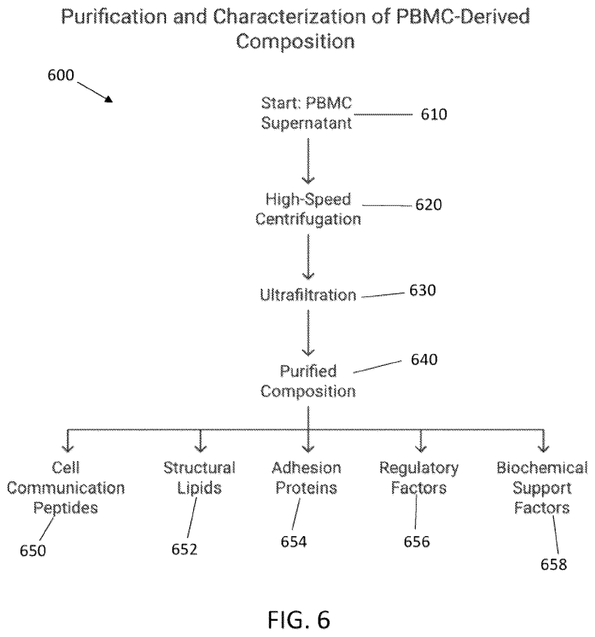

FIG. 6 is a diagram 600 showing purification and characterization of the PBMC-Derived composition and illustrates the purification and characterization process of the PBMC-derived composition, detailing its sequential refinement through high-speed centrifugation and ultrafiltration. The final purified composition retains essential biomolecules, including structural lipids, adhesion proteins, regulatory factors, biochemical support molecules, and cell communication peptides. This standardized process ensures high purity, functional stability, and bioactivity retention for biomedical and diagnostic applications.

### Breakdown of Key Stages in the Purification Process:

Start: PBMC Supernatant 610—Initial extraction of bioactive molecules from PBMC cultures.
High-Speed Centrifugation 620 Removes cellular debris and unwanted macromolecules.
Ultrafiltration Selectively 630 retains target biomolecules while eliminating impurities.
Purified Composition 640—Final bioactive formulation with enhanced purity and reproducibility.

As shown in FIG. 6, biomolecular components in the purified composition include cell communication peptides 650 to facilitate intercellular signaling, structural lipids 652 to contribute to membrane stability and integrity, adhesion proteins 654 to regulate cell-cell interactions, regulatory factors 656 to modulate immune response and inflammation and biochemical support factors 658 to assist in metabolic and regenerative functions.

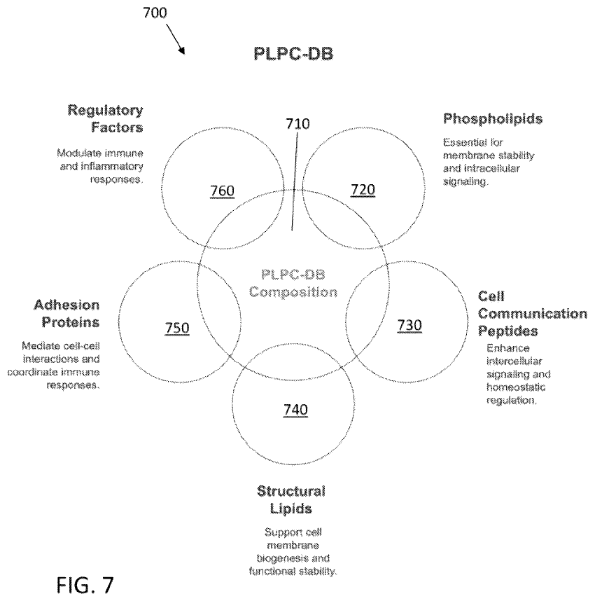

FIG. 7 is a diagram 700 showing the PLPC-DB composition 710 and functional biomolecular components. PLPC-DB is a highly purified phospholipoproteomic composition integrating essential biomolecules that contribute to cell signaling, immune modulation, and membrane stability. This schematic representation illustrates the key components of PLPC-DB and their functional roles, emphasizing its relevance in biomedical research, diagnostics, and therapeutic applications.

### Breakdown of Key Components in PLPC-DB Composition:

Phospholipids 720 are essential for membrane stability and intracellular signaling and serve as precursors for lipid-mediated biochemical pathways.
Cell communication peptides 730 enhance intercellular signaling and support homeostatic regulation and play a role in modulating cellular responses to environmental stimuli.
Structural lipids 740 support cell membrane biogenesis and functional stability and are essential for cell integrity and metabolic functions.
Adhesion proteins 750 mediate cell-cell interactions and coordinate immune responses and facilitate extracellular matrix communication and tissue structuring.
Regulatory factors 760 modulate immune and inflammatory responses and play a critical role in maintaining cellular balance and immune tolerance.

Scope and Adaptability of the Purification Process: Since multiple variations, modifications, and optimizations can be applied to different embodiments of the invention, this purification method is designed to be:
Flexible and adaptable, allowing integration into various molecular research and industrial applications.
Compliant with international regulatory frameworks, ensuring standardization in multicenter studies.
Scientifically robust and scalable, allowing for refinements and optimizations based on technological advancements in molecular purification.

One asect is directed to a phospholipoproteomic composition, characterized by the presence of functional phospholipids, bioactive proteins, and immunomodulatory cytokines derived from a supernatant of peripheral blood mononuclear cells (PBMCs). The phospholipoproteomic composition has a purity level exceeding 99% and is obtained through a multi-stage purification process including high-speed centrifugation, ultrafiltration and optimized freeze-drying. The high-speed centrifugation is performed at a speed of ≥10,000 RPM and is for molecular fractionation. Ultrafiltration at 1 to 50 kDa is for selective retention of biomolecules. Optimized freeze-drying ensures structural stability ≥24 months without the need for refrigeration. The phospholipoproteomic composition exhibits inter-batch variability of <2%, favoring consistency and reproducibility.

The phospholipoproteomic composition may be characterized by an optimized combination of phospholipids, bioactive proteins, cytokines and functional peptides, which have been studied in relation to structural stability, cell signaling and immunological modulations in biological environments. The phospholipoproteomic composition may be characterized by the presence of molecules that have been studied in relation to immune activation processes, reorganization of the cellular environment and metabolic regulation in specific biological systems. The phospholipoproteomic composition may be characterized by its optimized administration profile, favoring stability, bioaccessibility and molecular distribution in different biological models. The phospholipoproteomic composition may be characterized by the presence of biomolecules that have been studied in the context of immune regulation and cell regeneration mechanisms in different biological models. A process of manufacturing the composition may be characterized by an optimized manufacturing process, developed to promote stability, biological activity and reproducibility, through the implementation of advanced purification and quality control strategies. The composition may be characterized by the implementation of a quality and biological safety validation system, focused on purity, stability and bioactivity, through specialized controls in each production phase. The composition may be characterized by the presence of biomolecules that have been analyzed in the context of their interaction with immuno-oncological strategies in experimental models, exploring their relationship with cell activation mechanisms, modulation of immune signaling and metabolic regulation. The composition may be characterized by the presence of biomolecules that have been evaluated in the context of modulation of the immune response based on an immunological profile of an individual, allowing exploration of dynamic adjustments in therapeutic strategies. The composition may be characterized by the presence of biomolecules that have been evaluated in the context of modulation of chronic inflammatory processes and immunological balance in models of metabolic, autoimmune and neuroinflammatory diseases.

One aspect of a purified phospholipoproteomic composition is characterized by the presence of functional phospholipids, bioactive proteins and immunomodulatory cytokines, derived from the supernatant of peripheral blood mononuclear cells (PBMCs) or selected cell lines.

### 1. Purification and Stability Processes

The composition reaches purity levels greater than 99%, through:
The process methodology disclosed herein integrates high-speed centrifugation, ultrafiltration (1-50 kDa), and optimized freeze-drying, now further extended to immunological subset characterization and bioaccessibility profiling.
Centrifugation at ≥10,000 RPM for molecular fractionation.
Ultrafiltration (1-50 kDa) for selective retention of biomolecules.
Optimized freeze-drying, ensuring structural stability ≥24 months without the need for refrigeration.
Inter-batch variability <2%, favoring consistency and reproducibility.

### 2. Scientific and Biomedical Applications

In experimental studies, the composition has been used in the analysis of interactions between the tumor microenvironment and the immune system.
Its molecular profile has been evaluated in contexts related to inflammatory regulation mechanisms and immunological adaptation.
cellular repair and optimization of metabolic functions has been described.

### 3. Variability and Adaptation According to Cellular Source

An adaptable bioactive profile has been developed from different cell lines, adjusting the purification parameters.
Specific formulations have been documented aimed at:
   Regenerative lines: Applications in tissue studies and inflammatory modulation.
   Tumor lines: Evaluations in immunotherapies based on neoantigens.
   Immunological lines: Development of precision immunomodulatory compounds.

### 4. Dynamic Optimization Through Real-Time Monitoring

Flow cytometry used for molecular characterization in each purification series.
AI-based parameter tuning with open access (AlphaFold, RosettaFold).
Iterative refinement process, ensuring precision in the bioactive composition.

### 5. Preservation and Optimized Bioaccessibility

Freeze-drying under controlled pressure, favoring the preservation of the molecular structure.
Microaggregation phospholipid, contributing to greater solubility and distribution in biological models.

### 6. Process Integrity Assurance During High-Speed Centrifugation

The upstream separation of the phospholipoproteomic composition is conducted through a high-speed centrifugation process operating within the 10,000-25,000 RPM range, which enables precise biomolecular stratification without compromising the structural or functional integrity of the active constituents. To this end, the system integrates:
(a) Short-duration, high-efficiency cycles to minimize cumulative mechanical stress;
(b) Real-time thermal regulation to prevent temperature-induced denaturation or aggregation;
(c) Shear-limiting acceleration and deceleration profiles to preserve vesicle and protein complex architecture; and
(d) A dynamic, AI-assisted feedback loop that continuously adjusts RPM based on physicochemical profiles and density gradients.
This integrated control architecture ensures that all biomolecular fractions-including phosphatidylcholine, phosphatidylserine, sphingomyelin, NAMPT, TIGAR, HLA-A, integrins, tetraspanins, and regulatory peptides-retain their functional conformation, bioactivity, and purity exceeding 99%, even under industrial-scale ultracentrifugation scenarios required for advanced separation of complex vesicular structures.

### Molecular Structure and Bioactive Components:

Phospholipoproteomic composition characterized by an optimized combination of phospholipids, bioactive proteins, cytokines and functional peptides, which have been studied in relation to structural stability, cell signaling and immunological modulations in biological environments.

### 1. Functional Phospholipids:

Phosphatidylcholine (PC): 8,000-15,000 μg, identified in processes associated with membrane integrity and cell signaling;
Phosphatidylserine (PS): 5,000-10,000 μg, involved in cell recognition and phagocytosis mechanisms in different cellular environments;
Sphingomyelin: 3,500-8,500 μg, evaluated in relation to cellular homeostasis and regulation of immunological interactions.

### 2. Bioactive Proteins

NAMPT (1,500-3,000 μg): Related to mitochondrial metabolic processes and activity in specific cellular environments.
TIGAR (1,000-2,500 μg): Analyzed in oxidative stress and cell viability studies.
HLA-A (2,500-4,500 μg): Described in the context of antigen presentation and activation of specific lymphocytes.
Integrins α4β1 and αLβ2: Identified in cell adhesion and migration processes.
Tetraspanins CD81/CD63: Associated with regulation of intercellular communication.

### 3. Immune Regulatory Factors

IFN-γ (500-1,200 pg): Evaluated in processes that involve activation of cellular subpopulations in response to specific stimuli.
IL-12 (300-800 pg): Described in its interaction with cellular differentiation in certain immunological microenvironments.
CCL22 (200-600 pg): Involved in dendritic cell mobilization in cell migration studies.
CXCL10 (250-750 pg): Related to cellular infiltration phenomena in specific environments.
IL-15 (400-1,000 pg): Analyzed in the context of lymphocyte persistence and regulation under experimental conditions.

### 4. Functional Peptides

SDF-1 (200-700 pg): Studied for its interaction with mesenchymal stem cells in biological models.
TIMP-1 (100-300 pg): Evaluated in its relationship with metalloproteinases and extracellular matrix stability.
CD80/CD86: Described in the context of cellular activation in immunological studies.
Galectin-3: Identified in cell adhesion and proliferation processes in experimental environments.

### 5. Optimized Pharmaceutical Formulation

Lyophilization that allows immediate reconstitution and prolonged stability.
Stable format without the need for refrigeration, facilitating its integration into different study and application schemes.
   Mechanisms Associated with Immune Modulation:
   Phospholipoproteomic composition characterized by the presence of molecules that have been studied in relation to immune activation processes, reorganization of the cellular environment and metabolic regulation in specific biological systems.
   1. Interaction with Antigenic Activation Processes and Adaptive Response:
HLA-A (2,500-4,500 μg): Participates in the presentation of antigens in immunocompetent cells;
CD80/CD86 (100-500 pg): Identified in costimulatory signaling studies in T cells;
IL-12 (300-800 pg): Related to the differentiation of certain cell subtypes and production of immunological mediators.
   2. Factors Associated with the Regulation of the Cellular Microenvironment:
IFN-γ (500-1,200 pg): Tested in models exploring immune cell activation in different environments;
CXCL10 (250-750 pg): Related to the mobilization of specific cell populations in experimental studies;
TGF-β and IL-10 modulation: Evaluated in the context of immune signaling under certain conditions.

### 3. Metabolic Regulation and Sustainability of Cellular Response:

NAMPT (1,500-3,000 μg): Involved in the maintenance of energy metabolic pathways in activated cells;
TIGAR (1,000-2,500 μg): Studied in relation to the reduction of oxidative stress in immune cells;
IL-15 (400-1,000 pg): Described in studies on the persistence and activation of specific cell populations.
   4. Differentiation from Other Immunological Strategies:
The composition is not restricted to a single immune receptor, but can interact with multiple pathways;
The compositionhas been evaluated in models without requiring genetic manipulation or ex vivo cell expansion applied to patients;
Its molecular profile allows the activation of multiple immunological pathways without depending on a single mechanism.
   5. Compatibility with Combination Therapies and Personalized Strategies:
The compositionhas been explored in studies evaluating its effect on the persistence and functionality of advanced cellular therapies;
In experimental models, its impact on the response to radiotherapy and chemotherapy has been analyzed;
Its profile allows the adjustment of therapeutic strategies based on individual immunological biomarkers.

### Optimized Management and Bioaccessibility:

Phospholipoproteomic composition according to claim 2, characterized by its optimized administration profile, favoring stability, bioaccessibility and molecular distribution in different biological models.

### 1. Non-Invasive Administration Routes:

Endonasal/Mucosal: Evaluated in studies where its uptake by nasal dendritic cells and mucosal-associated lymphoid tissue (MALT) has been analyzed;
Sublingual/Transmucosal: Its absorption at the level of sublingual capillaries has been described in bioavailability studies, minimizing enzymatic degradation;
Transdermal/Intradermal: Investigated in contexts where its interaction with Langerhans and dermal dendritic cells has been explored, favoring progressive exposure to biological mediators.

### 2. Injectable Administration and Systemic Distribution:

Intravenous (IV): Analyzed in biodistribution studies with immediate systemic administration;
Intramuscular (IM)/Subcutaneous (SC): Evaluated in models with sustained release and prolonged activation;
Intratumoral/Intranodal: Described in research on its localization in specific cellular microenvironments.

### 3. Formulation Strategies for Pharmacokinetic Optimization:

nanoformulations: Use of phospholipid vesicles to improve stability and molecular distribution profile;
Rapid Reconstitution Lyophilization: Studies have demonstrated its stability at room temperature and immediate activation;
Controlled release in specific tissues: In preclinical models, its encapsulation has been explored to optimize exposure without systemic adverse effects.
   4. Differentiation from Other Management Strategies:
The compositiondoes not require genetic manipulation or ex vivo cell expansion, allowing greater versatility in its use;
adapt to different administration profiles has been described, adjusting to specific bioavailability needs;
In comparative studies, its formulation has been analyzed in relation to multifunctional immune responses without dependence on a single epitope.
   Immune Regulation and Processes Associated with Cellular Regeneration:
   Phospholipoproteomic composition characterized by the presence of biomolecules that have been studied in the context of immune regulation and cell regeneration mechanisms in different biological models.

### 1. Regulation of Immune Processes in Models of Autoimmune and Chronic Diseases:

CD80/CD86 (100-500 pg): Analyzed in the context of regulatory T cell (Treg) expansion in experimental settings;
CCL22 (200-600 pg): Related to dendritic cell migration processes and regulation of immune tolerance in cell interaction studies;
IL-6 and TNF-α: Evaluated in studies on inflammatory processes without affecting immune surveillance.
   2. Factors Associated with Regeneration Processes in Models of Cellular Degeneration and Traumatic Injuries:
SDF-1 (200-700 pg): Described in studies of mesenchymal stem cell mobilization in specific microenvironments;
TIMP-1 (100-300 pg): Evaluated in its relationship with the modulation of metalloproteinases and preservation of the extracellular matrix;
Mechanisms related to fibrosis reduction and activation of cellular repair processes have been explored in certain experimental models.
   3. Evaluation of Factors Associated with Chronic Inflammation and Cellular Metabolism:
NAMPT (1,500-3,000 μg): Analyzed in the context of metabolic mechanisms and immunological interactions in cellular processes;
Galectin-3 (50-200 pg): Described in the study of cell adhesion and modulation of inflammatory responses;
SASP regulation: Evaluated in studies on the modulation of IL-1β, IL-6 and TNF-α in chronic disease models.

### 4. Applications in Longevity and Cell Regeneration Models:

TIGAR (1,000-2,500 μg): Studied in mitochondrial protection processes and regulation of oxidative stress in specific cellular environments;
Mechanisms related to inflammatory regulation and cellular regeneration in healthy aging have been explored in preclinical studies;
   5. Differentiation from Conventional Immune Modulation and Regenerative Strategies:
systemic immunosuppression effects have been observed, allowing a more selective focus on inflammatory regulation;
Unlike stem cell-based strategies, formulations with purified biomolecules and guaranteed stability have been evaluated;
In validation studies, its molecular profile has been explored in multiple metabolic and immunological pathways without restriction to a single molecular target.

### Biopharmaceutical Production Process and Optimization:

Phospholipoproteomic composition characterized by an optimized manufacturing process, developed to promote stability, biological activity and reproducibility, through the implementation of advanced purification and quality control strategies.

### 1. Cellular Production and Secretion of Biomolecules:

Expansion and differentiation of PBMCs or selected cell lines under controlled conditions;
Selective induction aimed at optimizing the secretion of bioactive proteins and functional phospholipids;
Configuration of the cell type based on different experimental applications (regenerative, immunomodulatory or oncological).

### 2. Advanced Purification and Purity Control:

Centrifugation ≥10,000 RPM and ultrafiltration (1-50 kDa) for selective fractionation of biomolecules;
Purity validation by mass spectrometry and HPLC, reaching levels of active biomolecules greater than 99%;
Endotoxin reduction <0.25 EU/mg, meeting biological compatibility standards.
   3. Dynamic Optimization with Real-Time Monitoring:
Flow cytometry and advanced molecular spectroscopy techniques, allowing detailed characterization in each purification series;
Implementation of open-access AI algorithms (AlphaFold, RosettaFold) for structural analysis and dynamic parameter tuning;
Iterative process, favoring precision in the final bioactive composition.

### 4. Process Configuration According to Cell Type:

Tuning the cellular secretome based on specific applications;
Tumor lines for exploration in adaptive immunotherapy;
Regenerative lines in cell repair study models;
Purification adjustments to obtain differentiated bioactive profiles.

### 5. Preservation and Bioaccessibility Strategies:

Freeze-drying under controlled pressure, preserving the molecular structure;
Microaggregation phospholipid, optimizing solubility and biodistribution in biological models;
Stable format without the need for cryopreservation, allowing immediate availability.

### 6. Quality Control and Consistency in Production:

evaluation in each batch, ensuring functionality in controlled biological environments;
Inter-lot variability less than 2%, ensuring uniformity in production and industrial scalability.
   7. Differentiation from Other Biotherapies:
The compositiondoes not require genetic manipulation or autologous cell culture, simplifying regulatory processes;
Stable format without cryopreservation, favoring logistics and storage:
Lyophilization for rapid reconstitution, ensuring immediate availability under controlled conditions.
   Quality assurance and biological safety validation:
   Phospholipoproteomic composition according to claim 6, characterized by the implementation of a quality and biological safety validation system, focused on purity, stability and bioactivity, through specialized controls in each production phase.

### 1. Validation of Purity and Bioactivity:

Endotoxin Assay (LAL): Assessed to ensure levels below 0.25 EU/mg;
Structural confirmation by mass spectrometry and liquid chromatography (HPLC);
Quantification of key biomolecules (HLA-A, NAMPT, IL-12, IFN-γ) using ELISA and Western Blot techniques.

### 2. Stability and Storage Control:

Stability studies have demonstrated structural preservation for ≥24 months without the need for refrigeration;
Monitoring techniques have been implemented to prevent the formation of protein aggregates and loss of bioactivity under prolonged storage conditions.

### 3. Microbiological Safety and Contaminant Removal:

Sterile filtration using 0.22 μm membranes, evaluated in advanced purification processes;
Tests for the detection of viral agents and mycoplasmas, in compliance with biosafety standards;
Validation of osmolarity and pH in optimal physiological ranges, ensuring compatibility in your formulation.
   4. Differentiation from Other Biopharmaceuticals:
The compositiondoes not require cell viability validation or genetic manipulation, optimizing its integration into different application models;
The compositionhas been developed without relying on recombinant proteins or chemical modifications that alter its natural stability;
Inter-batch variability of <2% has been documented, ensuring consistency and reproducibility in industrial-scale manufacturing processes.

### Optimization of Immuno-Oncological Strategies:

Phospholipoproteomic composition characterized by the presence of biomolecules that have been analyzed in the context of their interaction with immuno-oncological strategies in experimental models, exploring their relationship with cell activation mechanisms, modulation of immune signaling and metabolic regulation.

1. Evaluation in Synergy with Immune Checkpoint Inhibitors:
Co-administration with anti-PD-1, anti-PD-L1 and anti-CTLA-4: Their impact has been explored in studies of cytotoxic lymphocyte persistence and activation;
Modulation of immunosuppressive signals: Studies have analyzed the regulation of TGF-β and IL-10 in specific tumor environments;
Optimization of antigen presentation: Its interaction with HLA-A and CD80/CD86, related to immunological recognition, has been evaluated.
   2. Interaction Analysis with Adoptive Therapies (CAR-T, NK):
Regulation of NAMPT and TIGAR: They have been explored in relation to mechanisms of preservation of cellular metabolism in activated lymphocytes;
Modulation of CXCL10 and CCL22: Analyzed in studies of lymphocyte migration and positioning in specific cellular environments;
Regulation of IL-12 and IFN-γ: Evaluated in the context of inflammatory signaling and effects on immune toxicity.
   3. Compatibility with Radiotherapy and Chemotherapy:
Tumor radiosensitization studies: Its relationship with immunogenicity induced by radiological damage has been explored;
Reduction of chemotherapy-induced immunosuppression: Functional protection mechanisms of the immune system have been evaluated under experimental conditions;
Regulation of IL-15 and VEGF-A: Analyzed in hematopoietic reconstitution processes post-cytotoxic treatment.
   4. Differentiation from Other Immunotherapeutic Strategies:
The composition is not restricted to a single immune checkpoint, as The compositioninteracts with multiple pathways in experimental models;
genetic manipulation or ex vivo cell expansion have been explored;
Its metabolic and immunological profile has been analyzed without evidence of systemic toxicity in preclinical studies.

### Personalized Immunotherapy and Therapeutic Adaptation:

Phospholipoproteomic composition characterized by the presence of biomolecules that have been evaluated in the context of modulation of the immune response based on the individual's immunological profile, allowing exploration of dynamic adjustments in therapeutic strategies.

### 1. Evaluation of Modulation Based on Immunological Biomarkers:

Analysis of immune activation in studies related to HLA-A, IL-12, IFN-γ, CXCL10 and SDF-1;
Adaptation to tumor mutational burden (TMB) and exploration of its interaction with the immune microenvironment;
Real-time immunophenotyping, allowing strategies to be adjusted based on specific cellular profiles.

### 2. Application in Tumor Models Refractory to Conventional Immunotherapies:

Evaluation of immune reactivation in PD-1/PD-L1 and CTLA-4 resistant models;
Induction of immune infiltration in low immunogenicity tumors, studied in preclinical models;
Exploration of metabolic regeneration in lymphocytes with NAMPT and TIGAR depletion.
   3. Compatibility with Advanced Cell Therapy Strategies:
Optimization of CAR-T and NK cells, investigated in terms of persistence and functionality in experimental models;
Interaction with epigenetic modulators, explored in the context of regulation of antigen presentation;
Dose adjustment and strategies based on predictive biomarkers, analyzed in different immunological profiles.
   4. Differentiation from Conventional Immunotherapeutic Strategies:
The composition is not restricted to a single molecular target, allowing its exploration in heterogeneous tumors;
Studies have analyzed its impact without compromising systemic immune surveillance, differentiating The compositionfrom conventional immunosuppressants;
Its profile has been evaluated in terms of standardization and reproducibility, differentiating The compositionfrom approaches based on cell therapies or exosomes.

### Regulation of Chronic Inflammatory Processes:

Phospholipoproteomic composition according to claim 5, characterized by the presence of biomolecules that have been evaluated in the context of modulation of chronic inflammatory processes and immunological balance in models of metabolic, autoimmune and neuroinflammatory diseases.

### 1. Evaluation of Inflammatory Processes in Models of Immune-Mediated and Neurodegenerative Diseases:

Regulation of IL-6, TNF-α and IFN-γ in studies on ulcerative colitis, Crohn's disease and multiple sclerosis;
Modulation of IL-17A, IL-10 and TGF-β, analyzed in the context of neuroinflammatory processes in experimental models;
Interaction with SDF-1/CXCR4 and VEGF-A, explored in studies on preservation of blood-brain and epithelial barriers.

### 2. Optimization of Immune Tolerance Without Induction of Systemic Immunosuppression:

Expansion of regulatory T lymphocytes (Treg) through CD80/CD86, studied in models of immune regulation;
Modulation of M1/M2 balance in macrophages, evaluated in studies on inflammatory homeostasis;
Exploration of TIMP-1 and MMP-9 in tissue remodeling processes and fibrosis prevention.

### 3. Applications in Models of Systemic and Metabolic Inflammatory Diseases:

Regulation of B lymphocytes in studies on psoriatic arthritis and systemic lupus erythematosus;
Analysis of metabolic inflammation in models of obesity and metabolic dysregulation;
Exploration of mechanisms of modulation of hepatic inflammation and fibrosis in cirrhosis and non-alcoholic steatohepatitis (NASH).
   4. Differentiation from Conventional Anti-inflammatory Strategies:
systemic immunosuppression has been evidenced, which is different from traditional immunomodulators;
Mechanisms other than IL-6 or TNF-α modulators have been explored, allowing interaction with multiple immunological pathways;
Immune homeostasis profiles have been analyzed without the adverse effects of conventional anti-inflammatory drugs.

### 5. Exploration in Models of Longevity and Cellular Regeneration:

Regulation of the senescence-associated secretory profile (SASP), analyzed in cellular aging studies;
Optimization of mitochondrial metabolism and reduction of oxidative stress, explored in relation to NAMPT and TIGAR;
Restoration of inflammatory profiles in aging models, aligned with cell regeneration strategies.

Another aspect of the composition is directed to a phospholipoproteomic composition, characterized by the presence of functional phospholipids, bioactive proteins, and immunomodulatory cytokines derived from a supernatant of peripheral blood mononuclear cells (PBMCs) or selected cell lines. The composition: (a) has a purity level exceeding 99%, (b) is obtained through a multi-step purification process including centrifugation, ultrafiltration with molecular weight cut-offs between 1-50 kDa, and lyophilization, (c) remains stable at ambient conditions for at least 24 months, and (d) exhibits inter-batch variability below 2%, ensuring consistency and reproducibility across production cycles.

The phospholipoproteomic composition may include of claim 1, comprising: (a) selected phospholipids, including phosphatidylcholine, phosphatidylserine, and sphingomyelin, associated with membrane integrity, cell signaling, and immunological interactions; (b) bioactive proteins, including NAMPT, TIGAR, and HLA-A, as well as integrins α4β1, αLβ2, and tetraspanins CD81/CD63, involved in metabolic regulation, antigen presentation, and immune cell migration; (c) immunomodulatory factors such as IFN-γ, IL-12, CCL22, CXCL10, and IL-15, identified through molecular profiling and characterized for their role in immune coordination; and (d) regulatory peptides including SDF-1, TIMP-1, CD80/CD86, and Galectin-3, contributing to extracellular matrix regulation and adaptive immune responses; and (c) a lyophilized pharmaceutical format, designed for rapid reconstitution and structural stability under ambient conditions.

The molecular components may be associated with: (a) antigen presentation via HLA-A and co-stimulation through CD80/CD86; (b) immune cell activation and polarization involving IL-12, IFN-γ, CXCL10, and CCL22; (c) metabolic regulation through NAMPT and TIGAR, supporting oxidative balance; (d) IL-15-associated persistence of lymphocytes in controlled biological environments; and (e) immune checkpoint interactions involving regulatory cytokines such as TGF-β and IL-10. These interactions have been characterized through molecular profiling in experimental systems. The phospholipoproteomic composition may be formulated for administration through parenteral, mucosal, or transdermal routes, comprising: (a) non-invasive delivery options including sublingual, endonasal, transmucosal, transdermal, and intradermal application; (b) injectable routes including intravenous (IV), intramuscular (IM), subcutaneous (SC), intratumoral, and intranodal administration; and (c) formulation enhancements incorporating phospholipid-based nanoassemblies, lyophilized powder reconstitution, and controlled-release encapsulation systems. Bioaccessibility and molecular distribution parameters have been evaluated in biological systems without implying therapeutic claims. The selected molecular subsets may be associated with immune regulation and extracellular matrix modulation, including: (a) CD80/CD86 and CCL22, linked to co-stimulation and dendritic cell migration; (b) IL-6, TNF-α, and Galectin-3, involved in immune regulation and cell adhesion; (c) TIMP-1 and SDF-1, contributing to extracellular matrix stabilization and cellular recruitment; and (d) NAMPT and TIGAR, associated with metabolic homeostasis and oxidative stress modulation. These elements have been characterized in controlled biological models without reference to therapeutic outcomes.

The structural, immunological, and stability-related features of the present composition are supported by experimental methodologies and analytical strategies disclosed in the parent application. These technical elements have been subsequently reinforced through four independent Q1 peer-reviewed publications authored by the present inventor, which expand upon the foundational principles without modifying the original disclosure. Such external literature, while not formally incorporated, supports the broader scientific relevance and reproducibility of the composition under examination.

A process for producing the phospholipoproteomic composition includes: (a) culturing PBMCs or selected cell lines under sterile conditions with targeted stimulation; (b) purifying the supernatant by centrifugation (10,000-25,000 RPM) and ultrafiltration (3-50 kDa) to selectively retain phospholipoproteins, immune regulatory proteins, and optionally extracellular vesicles; (c) lyophilizing under controlled conditions to preserve molecular integrity; (d) profiling bioactive fractions in real-time or near-real-time via analytical methods; and (e) iteratively adjusting purification parameters via AI-assisted feedback. Operational settings, including time, temperature, and membrane selection, remain proprietary as industrial know-how, .including time, temperature, membrane configuration, and other critical control variables that may be internally adjusted and preserved as proprietary know-how.

The phospholipoproteomic composition may include a quality and safety validation system ensuring: (a) endotoxin control (<0.25 EU/mg), structural integrity (HPLC, mass spectrometry), and sterility verification (0.22 μm filtration); (b) quantification of bioactive molecules (NAMPT, HLA-A, IL-12, IFN-γ) using immunoassays; (c) stability, solubility, and formulation consistency monitored under defined specifications; and (d) batch-to-batch reproducibility with inter-lot variation maintained below 2%. The wherein molecular subsets may be analyzed in immuno-oncological models, including: (a) interaction with immune checkpoint blockade models; (b) antigen presentation via HLA-A, CD80/CD86, and modulation of TGF-β and IL-10; (c) NAMPT and TIGAR involvement in immune metabolic regulation; (d) CXCL10 and CCL22 in tumor infiltration; (c) IL-12 and IFN-γ in immune polarization; and (f) IL-15 and VEGF-A in vascular and recovery signaling. These interactions have been evaluated in controlled biological systems. All biological effects and molecular interactions described herein have been observed in controlled experimental systems or biological models and are not to be interpreted as therapeutic claims. This continuation does not seek to claim therapeutic use or indication, in accordance with 35 U.S.C. § 101 limitations. The molecular subsets may be evaluated for adaptive immunotherapy, including: (a) immune biomarker profiling, including HLA-A, IL-12, IFN-γ, CXCL10, and SDF-1; (b) modulation based on tumor mutational burden and immune infiltration indices; (c) integration with immunophenotyping to adjust formulation parameters; (d) compatibility with refractory tumor models and checkpoint blockade strategies; and (e) formulation refinement based on experimental immunoprofiling. Molecular subsets may be evaluated for inflammatory regulation and immune homeostasis, including: (a) modulation of IL-6, TNF-α, IFN-γ, IL-17A, IL-10, and TGF-β in experimental models; (b) structural contributions to barrier integrity via SDF-1/CXCR4 and VEGF-A; (c) involvement of CD80/CD86 and TIMP-1 in immune regulation and extracellular matrix stabilization; and (d) SASP-associated biomarkers and mitochondrial regulators including NAMPT and TIGAR; These molecular interactions have been studied in controlled biological systems without reference to clinical endpoints.

The scope of this invention is therefore not limited to the current configurations, as future enhancements and research applications can further refine the methodology, ensuring its continued relevance in biotechnology and applied life sciences.

The claims appended to this invention will define the full extent of its applicability, reinforcing its position as a leading innovation in biomolecular purification and high-purity bioinput development.

Reservation of Rights for Future Applications and Additional Claims: To the extent that the foregoing description and accompanying drawings disclose innovative subject matter beyond the scope of the following claims, such inventions are not dedicated to the public domain. The right to file additional applications covering such subject matter is expressly reserved.

This ensures that any new technological advancements, refinements, or novel embodiments derived from this invention can be protected under subsequent patent applications, preserving the intellectual property rights and exclusivity of the disclosed purification and biomolecular composition methodologies.

Scope of the Invention and Expansion of Patent Claims: Although the current set of claims is limited, it is essential to recognize that the scope of this invention extends far beyond the claims presented in this document. The present disclosure is intended to serve as a foundational patent application, with the expectation that additional, broader claims will be presented in a subsequent application to fully leverage the priority benefits of this submission.

By structuring the patent application in a strategic manner, this invention ensures:
a. Comprehensive protection of the innovative purification methodology and phospholipoproteomic composition.
b. The ability to expand intellectual property rights as new refinements, formulations, or use cases emerge.
c. Legal flexibility for filing divisional applications that may encompass further enhancements, manufacturing variations, and new biomedical applications.
   This provision ensures that future innovations derived from PLPC-DB remain protected and strategically positioned in the biotechnology and biomolecular research sectors.

Adaptability of the Invention and Legal Coverage: While this invention has been described with reference to specific embodiments, it is essential to acknowledge that further modifications, optimizations, and enhancements may be made without departing from the core principles and scope of the disclosure.

This application is structured to cover a wide range of potential variations, including:
a. New methodologies that build upon the core purification process, optimizing biomolecular fractionation, protein stability, and phospholipid preservation.
b. Additional applications in emerging research areas, including precision medicine, synthetic biology, and bioengineered therapeutics.
c. Alternative configurations of the process, allowing for customization in molecular research and commercial biomaterial development.

Furthermore, this application explicitly encompasses departures from the present disclosure that fall within:
a. The known or usual practice in the field of biomolecular purification and research.
b. The limits of the appended claims, ensuring comprehensive legal protection for the disclosed innovation.

By maintaining a broad, adaptable patent framework, this invention secures its position as a pioneering advancement in purified phospholipoproteomic bioinputs, ensuring scientific relevance, regulatory compliance, and long-term commercial viability.

We claim:

1. A phospholipoproteomic composition, characterized by the presence of:
functional phospholipids, bioactive proteins, and immunomodulatory cytokines derived from a supernatant of peripheral blood mononuclear cells (PBMCs) or selected cell lines, wherein the composition:
(a) has a purity level exceeding 99%;
(b) is obtained through a multi-step purification process including centrifugation, ultrafiltration with molecular weight cut-offs between 1-50 kDa, and lyophilization;
(c) remains stable at ambient conditions for at least 24 months; and
(d) exhibits inter-batch variability below 2%, ensuring consistency and reproducibility across production cycles.

2. The phospholipoproteomic composition of claim 1, comprising:
(a) selected phospholipids, including phosphatidylcholine, phosphatidylserine, and sphingomyelin, associated with membrane integrity, cell signaling, and immunological interactions;
(b) bioactive proteins, including NAMPT, TIGAR, and HLA-A, as well as integrins α4β1, αLβ2, and tetraspanins CD81/CD63, involved in metabolic regulation, antigen presentation, and immune cell migration;
(c) immunomodulatory factors such as IFN-γ, IL-12, CCL22, CXCL10, and IL-15, identified through molecular profiling and characterized for their role in immune coordination;
(d) regulatory peptides including SDF-1, TIMP-1, CD80/CD86, and Galectin-3, contributing to extracellular matrix regulation and adaptive immune responses; and
(e) a lyophilized pharmaceutical format, designed for rapid reconstitution and structural stability under ambient conditions.

3. The phospholipoproteomic composition of claim 1, wherein the molecular components are associated with:
(a) antigen presentation via HLA-A and co-stimulation through CD80/CD86;
(b) immune cell activation and polarization involving IL-12, IFN-γ, CXCL10, and CCL22;
(c) metabolic regulation through NAMPT and TIGAR, supporting oxidative balance;
(d) IL-15-associated persistence of lymphocytes in controlled biological environments; and
(e) immune checkpoint interactions involving regulatory cytokines such as TGF-β and IL-10;
wherein these interactions have been characterized through molecular profiling in experimental systems.

4. The phospholipoproteomic composition of claim 1, wherein molecular subsets have been analyzed in immuno-oncological models, comprising:
(a) interaction with immune checkpoint blockade models;
(b) antigen presentation via HLA-A, CD80/CD86, and modulation of TGF-β and IL-10;
(c) NAMPT and TIGAR involvement in immune metabolic regulation;
(d) CXCL10 and CCL22 in tumor infiltration;
(e) IL-12 and IFN-γ in immune polarization; and
(f) IL-15 and VEGF-A in vascular and recovery signaling:
wherein these interactions have been evaluated in controlled biological systems.
